(19) United States
(12) Patent Application Publication
(10) Pub. No.: US 2022/0415438 A1
(43) Pub. Date: Dec. 29, 2022

(54) Diagnosis of Malignancy Using Developmental Relationships and Machine Learning

(71) Applicant: Massachusetts Institute of Technology, Cambridge, MA (US)

(72) Inventors: Enrico Moiso, Cambridge, MA (US); Salil Garg, Wellesley, MA (US)

(21) Appl. No.: 17/852,156

(22) Filed: Jun. 28, 2022

Related U.S. Application Data
(60) Provisional application No. 63/216,091, filed on Jun. 29, 2021.

(51) Int. Cl. G16B 25/00; G16B 40/20; G16B 45/00; G06N 3/12

(52) U.S. Cl. CPC G16B 25/00; G16B 40/20; G16B 45/00; G06N 3/126

(57) ABSTRACT

A computer-implemented method and system uses a map which maps from gene expression data for a plurality of training tumors in a tumor atlas to gene expression data representing single cells derived from mammal samples in developmental stages in a single-cell atlas. The method and system: (A) use the map to extract, from the plurality of training tumors, a plurality of biological components, thereby generating, for each training tumor-biological component pair, a corresponding biological component score; and (B) construct, based on the two atlases and the map, a machine learning perceptron classifier that outputs a tumor type of an input tumor based on its gene expression data. The method and system may generate the map before using it. The method and system may apply the machine learning perceptron classifier to the input tumor's gene expression data to generate the tumor type of the input tumor.

### STATEMENT AS TO FEDERALLY SPONSORED RESEARCH

This invention was made with Government support under grant numbers NCI K08-CA237856, P30-CA14051, and R37-CA225655 awarded by NIH. The Government has certain rights in the invention.

### BACKGROUND

Diagnosis of malignancy relies on histopathological classification of tumor appearance, often alongside other features such as mutation profiling and clinical presentation. However, many tumors display a spectrum of heterogeneous appearances on histopathologic analysis, which may in part reflect unknown differences in their development. Heterogeneous tumor appearance can lead to diagnostic uncertainty, with either disagreement amongst pathologists, overdiagnosis, underdiagnosis, or inability to distinguish ‘gray zone’ cases between tumor types. Additionally, the cell type of origin for some cancers is unclear, and these malignancies are usually classified based on tissue of occurrence. Further, tumors can dedifferentiate, correlating with more aggressive behavior and complicating diagnostic identification. Cancers of unknown primary (CUPs) represent malignancies that are often particularly dedifferentiated and aggressive with poor survival rates. Lack of diagnostic information is one factor that complicates treatment of many cancers, including CUPs, which are usually treated using non-targeted therapies with harsh toxicities. Understanding developmental pathways dysregulated in malignancies is a major goal in cancer biology, and could enable targeted therapeutic interventions guided by more precise diagnosis tools.

Machine learning classifiers have shown promise as new tools when applied to image processing in radiology and histopathology. In previous work, researchers have trained machine-learning-based image classifiers to distinguish different types of cancer on the basis of their histopathological appearance. However, image classifiers can only detect visual features present in the histopathology slides, and such classifiers are sometimes subject to artifacts. Classifiers that use molecular features, such as gene expression, have great potential to aid in diagnosis through capturing non-visual information, and recent approaches have demonstrated value in combining visual and molecular features for tumor classification. Genetic data in particular can be used to train machine learning classifiers. However, classifiers trained on gene expression data have suffered from overfitting due to the high dimensionality of the input data which can span tens of thousands of protein-coding genes. Overfitting in turn results in poor predictive performance on new datasets. Alternatively, selecting small gene panels for measurement also reduces predictive power by not utilizing all the available information. A key challenge in utilizing gene expression data to build integrated diagnostic models remains how to reduce the number of features while extracting the most relevant information.

### SUMMARY

A computer-implemented method and system uses a map which maps from gene expression data for a plurality of training tumors in a tumor atlas to gene expression data representing single cells derived from mammal samples in developmental stages (e.g., prenatal developmental stages) in a single-cell atlas. The method and system: (A) use the map to extract, from the plurality of training tumors, a plurality of biological components, thereby generating, for each of the plurality of training tumors and each of the plurality of biological components, a corresponding biological component score representing the training tumor as a compilation of non-gene terms describing known biological programs; and (B) construct, based on the single-cell atlas, the tumor atlas, and the map, a machine learning perceptron classifier that outputs a tumor type of an input tumor based on gene expression data for the input tumor, wherein the input tumor is not among the plurality of training tumors.

Before using the map, the method and system may generate the map by: (C) receiving a single-cell atlas comprising gene expression data; (D) receiving a tumor atlas comprising gene expression data for a plurality of training tumors; and (E) generating the map, which maps from the gene expression data in the tumor atlas to the gene expression data in the single-cell atlas. The gene expression data in the single-cell atlas may represent, for example, organogenesis of a plurality of mammalian development trajectories.

The method and system may also apply the machine learning perceptron classifier to the gene expression data for the input tumor to generate the tumor type of the input tumor.

Other features and advantages of various aspects and embodiments of the present invention will become apparent from the following description and from the claims.

### BRIEF DESCRIPTION OF THE DRAWINGS

FIG. 1A is a dataflow diagram of a system for identifying the tumor type of an input tumor according to one embodiment of the present invention;

FIG. 1B is a dataflow diagram of a system for constructing a machine learning perceptron classifier for generating tumor type data in one embodiment of the present invention;

FIG. 1C is a dataflow diagram of a system for generating a map between a tumor atlas and a single-cell atlas according to one embodiment of the present invention;

FIG. 2A is a flowchart of a method performed by the system of FIG. 1 according to one embodiment of the present invention;

FIG. 2B is a flowchart of a method performed by the system of FIG. 1B according to one embodiment of the present invention;

FIG. 2C is a flowchart of a method performed by the system FIG. 1C according to one embodiment of the present invention;

FIGS. 3A-3B show an example of validation of a classifier using single-cell RNA sequencing data and tumor-normal mixing.

FIGS. 4A-4D show an example of developmental deconvolution capturing signals from normal and malignant cells.

FIGS. 5A-5B show an example of developmental deconvolution radar plots for eight randomly-chosen T-cells and normal cell types from single-cell RNA sequencing.

FIGS. 6A-6D show a high-level example of diagnosis of malignancy by developmental deconvolution and machine learning.

FIGS. 7A-7F show a visualization of the systematic correlation of a tumor atlas to developmental trajectories.

FIGS. 8A-8D show an example of developmental correlations between a tumor atlas, TCGA, and a single-cell atlas, MOCA.

FIGS. 9A-9B show an example of validation of a tumor atlas (TGCA) and a single-cell atlas (MOCA) correlation using human developmental trajectories.

FIGS. 10A-10B show an example of tumor-normal tissue type comparison for developmental trajectories.

FIG. 11 show an example of known tumor type and Spearman correlation coefficient for the rank order of trajectory correlation for institution specific tumor samples against the equivalent TCGA primary tumor type.

FIGS. 12A-12F show an example of developmental deconvolution of tumor samples.

FIGS. 13A-13C show an example of diagnosis of Cancer of Unknown Primary by a developmental multilayer perceptron classifier.

FIGS. 14A-14G show an example of construction and testing of a developmental multilayer perceptron classifier for tumor type.

FIG. 15 is a confusion matrix for an example developmental multilayer perceptron's performance.

### DETAILED DESCRIPTION

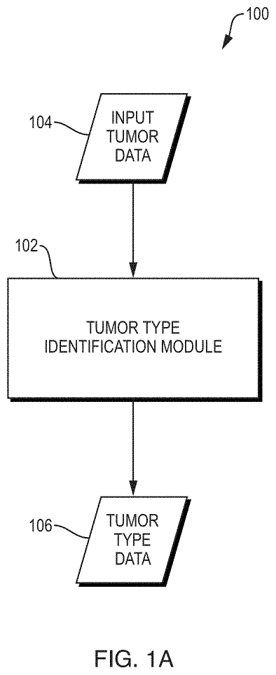

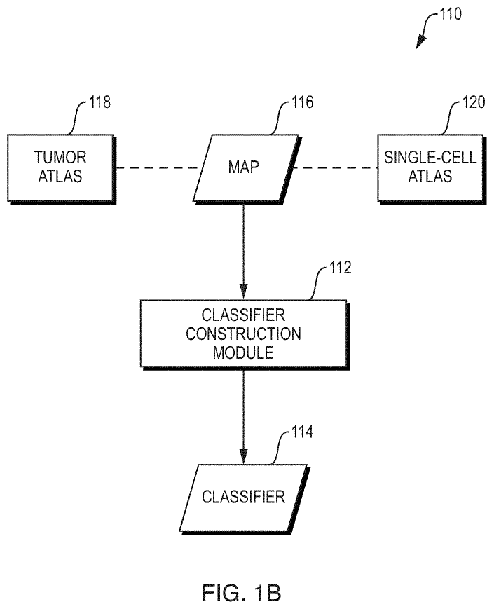

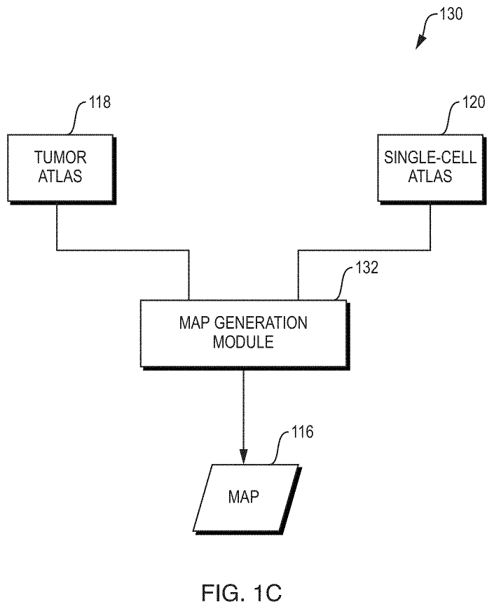

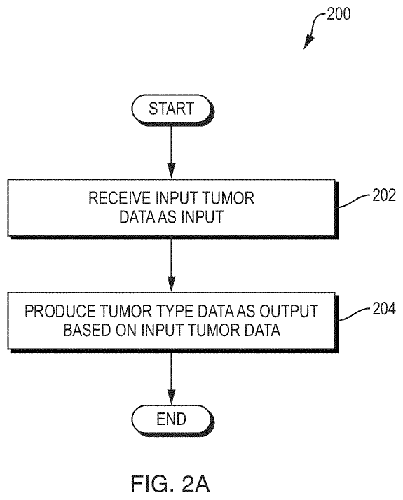

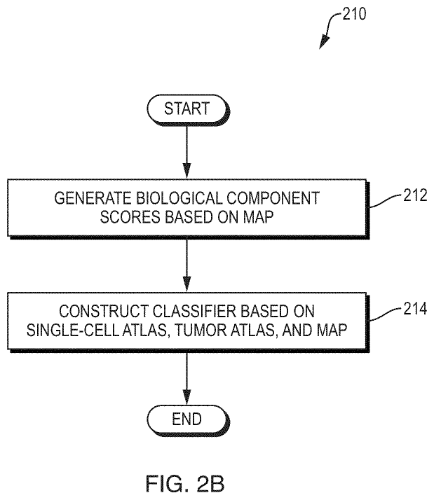

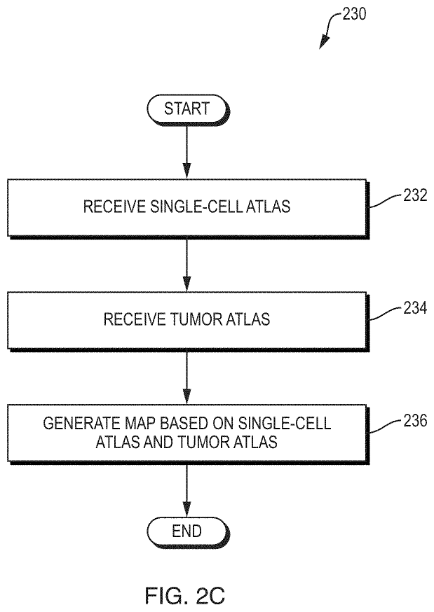

Referring to FIG. 1A, a dataflow diagram is shown of a system 100 for identifying the tumor type of an input tumor according to one embodiment of the present invention. Referring to FIG. 2A, a flowchart is shown of a method 200 performed by the system of FIG. 1A according to one embodiment of the present invention.

The system 100 includes a tumor type identification module 102 which receives, as input, data 104 representing the input tumor (FIG. 2A, operation 202). The tumor type identification module 102 produces, as output, based on the input tumor data 104, data 106 representing the tumor type of the input tumor (FIG. 2A, operation 204). The tumor type data 106 may or may not be accurate; in this sense the tumor type data 106 represents a prediction or estimate of the actual tumor type of the input tumor. The input tumor represented by the input tumor data may be a sample of a cancer of unknown primary (CUP).

Embodiments of the present invention may generate the tumor type data 106 in a variety of ways. For example, referring to FIG. 1B, a dataflow diagram is shown of a system 110 for constructing a machine learning perceptron classifier 114 that may be used by the tumor type identification module 102 to generate the tumor type data 106 in one embodiment of the present invention. Referring to FIG. 2B, a flowchart is shown of a method 210 performed by the system 110 of FIG. 1B according to one embodiment of the present invention. The method 210 of FIG. 2B may, for example, be performed before the method 200 of FIG. 2A, so that the classifier 114 may be used by the tumor type identification module 102 of FIG. 1A in the method 200 of FIG. 2A. As this implies, the methods

The system 110 includes a classifier construction module 112. As described in more detail below, the classifier construction module 112 constructs a classifier 114, which is capable of outputting data representing a tumor type of an input tumor. For example, the tumor type identification module 102 of FIG. 1A may include, or otherwise access, the classifier 114, and may use the classifier 114 to generate the tumor type data 106, which represents a tumor type of the input tumor represented by the input tumor data 104.

The classifier construction module 112 may construct the classifier 114 in any of a variety of ways. For example, the system 110 may include a tumor atlas 118 and a single-cell atlas 120, which may be used to produce a map 116 (as described in more detail below in connection with FIGS. 1C and 2C). The tumor atlas 118 may, for example, include gene expression data for a plurality of training tumors. Such gene expression data for the plurality of training tumors may, for example, include, for each of the plurality of training tumors: (1) gene sequencing data for the training tumor; and (2) a label indicating a type of cancer of the training tumor. The single-cell atlas 120 may, for example, include gene expression data representing single cells derived from mammal samples in developmental stages (e.g., prenatal developmental stages).

The map 116 may map from (some or all of) the gene expression data for the plurality of training tumors in the tumor atlas 118 to (some or all of) the gene expression data representing single cells in the single-cell atlas 120.

The system 110 may include a biological component score generation module 122, which may receive the map 116 produced from the tumor atlas 118 and the single-cell atlas 120, and use the map 116 to extract, from the plurality of training tumors in the tumor atlas 118, a plurality of biological components, thereby generating, for each of the plurality of training tumors and each of the plurality of biological components, a corresponding biological component score representing the training tumor as a compilation of non-gene terms describing known biological programs (such as a plurality of known developmental programs) (FIG. 2B, operation 212). The resulting biological component scores 124 are shown as the output of the biological component score generation module 122 in FIG. 1B.

The extraction of the plurality of biological components in operation 212 may, for example, include using the map 116 to deconvolute the plurality of training tumors into the plurality of biological components.

The classifier construction module 112 may construct the machine learning perceptron classifier 114 based on the map 116, which was constructed from the single-cell atlas 120 and the tumor atlas 118 (FIG. 2B, operation 214). The classifier 114 is capable of outputting a tumor type of an input tumor based on gene expression data for the input tumor. For example, after performing the method 210 of FIG. 2B to construct the classifier 114, embodiments of the present invention may perform the method 200 of FIG. 2A to apply the classifier 114 (which may be within the tumor type identification module 102) to gene expression data for an input tumor (e.g., the input tumor data 104) to generate the tumor type data 106, which represents a tumor type of the input tumor.

Note that the input tumor may not be among the plurality of training tumors; i.e., the classifier 114 can generate tumor types for tumors that were not in the tumor atlas 118 that was used to construct the classifier 114.

The tumor atlas 118 may take any of a variety of forms. For example, in some embodiments of the present invention, the tumor atlas 118 is, or includes, or is included within, some or all of a version of The Cancer Genome Atlas (TCGA).

The single-cell atlas 120 may take any of a variety of forms. For example, in some embodiments of the present invention, the single-cell atlas is, or includes, or is included within, a developmental atlas. Such a developmental atlas may, for example, be a single-cell organogenesis atlas. The single-cell atlas may, for example, include data representing normal development for each of a plurality of mammalian developmental trajectories. The single-cell atlas may, for example, be, or include, or be included within a version of the Mouse Organogenesis Cell Atlas (MOCA).

Embodiments of the present invention may generate the map 116 before performing the method 200 of FIG. 2A and/or before performing the method 210 of FIG. 2B. For example, referring to FIG. 1C, a dataflow diagram is shown of a system 130 for generating the map 116 in one embodiment of the present invention. Referring to FIG. 2C, a flowchart is shown of a method 230 performed by the system 130 of FIG. 1C according to one embodiment of the present invention. The method 210 of FIG. 2C may be performed before the method 210 of FIG. 2B, so that the map 116 may be used by the system 110 of FIG. 1B in the ways disclosed herein.

The system 130 includes a map generation module 132, which receives as inputs: (1) the single-cell atlas 120, which includes gene expression data representing organogenesis of a plurality of mammalian development trajectories (FIG. 2C, operation 232); and (2) the tumor atlas 118, which includes gene expression data for a plurality of training tumors (FIG. 2C, operation 234). The map generation module 132 generates the map 116 based on some or all of the single-cell atlas 120 and the tumor atlas 118 (FIG. 2C, operation 236). Such generation of the map 116 may be performed, for example, using normalization and rank-based, non-parametric approaches, such as Spearman correlation coefficient. The resulting map 116 maps from the gene expression data in the tumor atlas 118 to the gene expression data in the single-cell atlas 120.

Generating the map in operation 236 may include, for example, mapping each of the plurality of training tumors (in the tumor atlas 118) to at least one of the plurality of mammalian developmental trajectories in the single-cell atlas 120.

Having described various embodiments of the present invention at a high level, certain implementations of embodiments of the present invention will now be described in more detail.

### Tumor Datasets

It was described above that a tumor atlas 118 may be used and that the tumor atlas 118 may include gene expression data for a plurality of training tumors, such as gene sequencing data and a label indicating a type of cancer of the training tumor. The tumor atlas 118 may include The Cancer Genome Atlas (TCGA), non-TCGA sample cohort data, and/or institution-derived sample cohort data. TCGA contains bulk RNA sequencing data for 33 tumor and normal tissue types accompanied by diagnostic annotations. There are 10,388 samples, with 62 different sample types. Non-TCGA data may include tumor samples obtained from various cancer studies. Institution-derived data may be used for validation of methods in an independent sample set. Below, additional details are provided about these datasets.

### The Cancer Genome Atlas (TCGA) Data

The tumor atlas 118 may include, for example, The Cancer Genome Atlas (TCGA). For example, the coding gene expression profile [RNAseqV2_RSEM_genes_normalized_data_Level_3] and clinical information [Merge_Clinical.Level_1.2016012800.0.0] of TCGA samples (release 2016_02_28) may be systematically downloaded using firehose_get v 0.4.1 tool, from the Broad TCGA GDAC site (https://gdac.broadinstitute.org/). This contains the data and the analytic categories used by TCGA consortium, including for aggregating data for the following tumor types: COADREAD (COAD+READ), GBMLGG (GBM+LGG), KIDNEY (KICH+KIRC+KIRP), and STES (ESCA+STAD). A category LUNG may be created by aggregating LUAD and LUSC.

### Non-TCGA Sample Cohort Data

The tumor atlas 118 may also include, for example, data aggregated from various cancer studies. For example, a non-TCGA sample cohort may be created by aggregating data from cancer studies with gene expression profiles and clinical information retrieved from the Genomic Data Commons (GDC) Data Portal (https://portal.gdc.cancer.gov/). A merging of TCGA categories may allow incorporation of the maximum number of non-TCGA samples, as non-TCGA studies use slightly different diagnostic categories.

Institution-Derived Data, e.g. MGH Sample Cohort Data

The tumor atlas 118 may also include, for example, tumor data derived from a particular institution such as Massachusetts General Hospital (MGH). For example, in an embodiment, an MGH Sample Cohort may be created using samples from formalin fixed paraffin embedded (FFPE) tissue from cases seen in the Center for Integrated Diagnostics in the Department of Pathology at Massachusetts General Hospital either with known diagnosis (33 cases) or as cancers of unknown primary (52 cases). Total nucleic acid may be isolated from six scraped blank slides using clinically validated protocols.

RNA Extraction from FFPE Clinical Samples and RNA Sequencing Analysis of FFPE Clinical Samples

Tumor data derived from FFPE clinical samples may be obtained through RNA extraction and RNA sequencing processes. RNA extraction from FFPE clinical samples may involve the following steps: libraries may be prepared using a modified version of the Takara SMARTer Stranded Total RNA-Seq Kit—Pico Input Mammalian kit. In brief 100 ng of RNA at 10 ng/ul may be sonicated using RL230 Covaris sonicator (Covaris Inc) and resulting material may be confirmed using a Fragment Analyzer (Agilent). 10 ng of each sonicated sample may be prepared using the pico input kit as for FFPE samples using a 1:8 volume reduction on the STP MosquitoHV. Final libraries may be validated by Fragment Analyzer and qPCR prior to sequencing on a NovaSeq6000 S4 with 150 nt paired-end reads.

RNA-sequencing analysis of FFPE-derived clinical samples may be performed. Reads obtained from sequencing step may be processed as follows. A STAR reference genome using GENCODE v35 fasta and gtf files may be generated using STAR ‘genomeGenerate’. Next, fastq files may be aligned to the genome generated with STAR using two pass mapping (see mgh sequencing.sh for details on STAR parameters). This step may generate bam files compatible with RSEM gene expression calculation. An RSEM reference may be prepared using rsem-prepare-reference command. These files along with the bam files may be used to calculate gene expression with rsem-calculate-expression using parameters (—p 16, —bam, —paired-end, —no-bam-output, —forward-prob 0.5, and —seed 12345).

Gene expression measured in transcripts per million (TPM) may be used to assess the similarity with MOCA dataset. Homo Sapiens primary assembly v35_GRCh38.p13 genome and relative annotation GTF file (v35) may be downloaded from GENCODE website (https://www.gencodegenes.org/). STAR v2.7.1a alignment tools, RSEM v1.3.1, R v3.6.0 (https://www.R-project.org/) and Perl v5.24.1 may be used for gene expression analysis.

### Cancer Single-Cell Sequencing Data

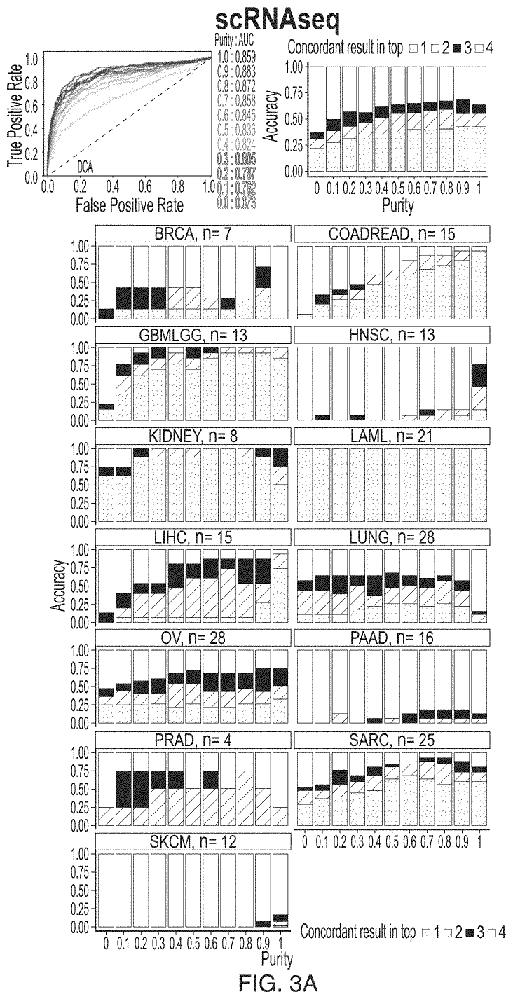

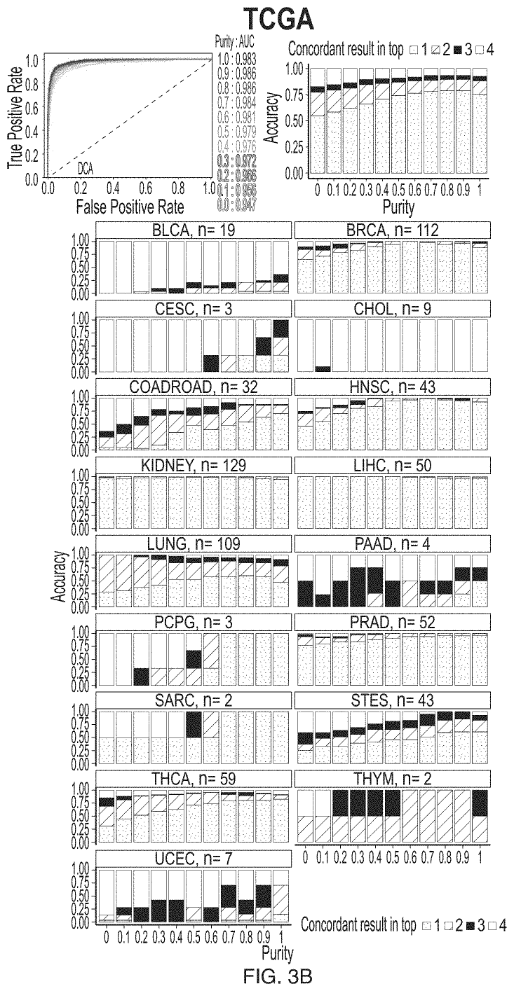

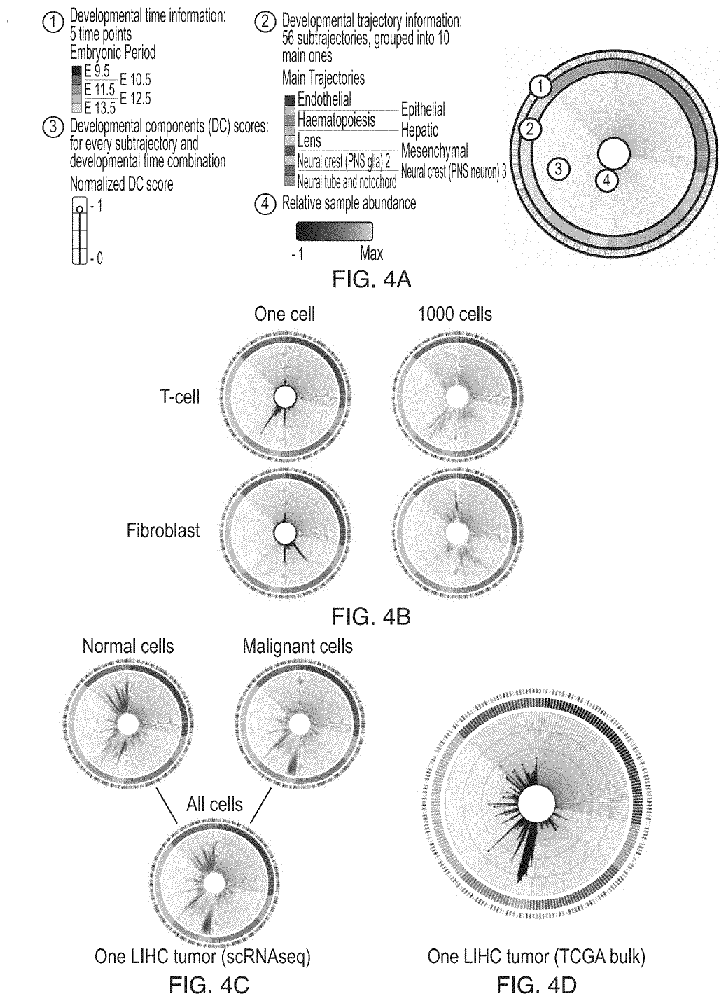

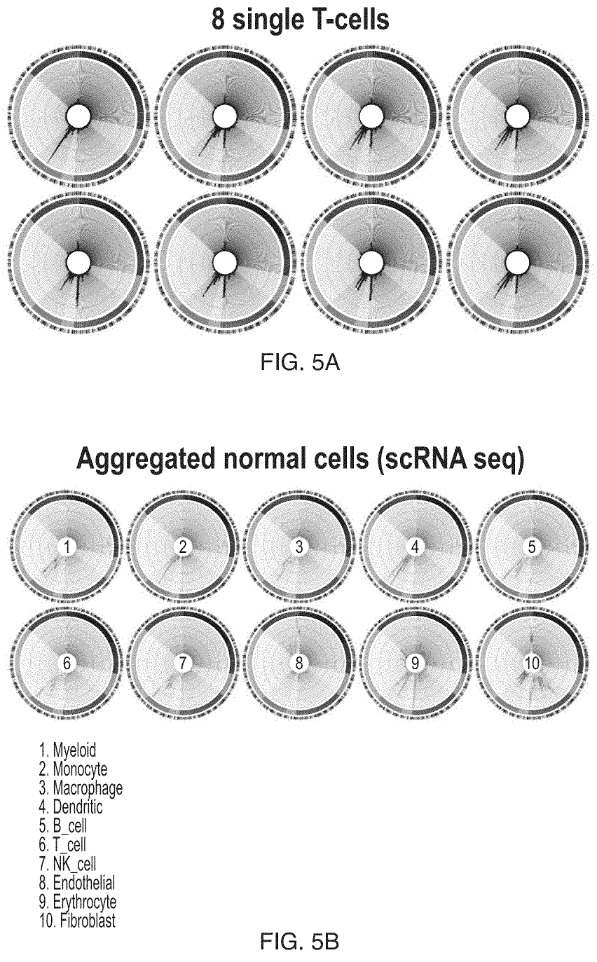

Tumor-related data 104 may also include cancer single-cell sequencing data. For example, cancer single-cell sequencing data may include the expression profile of normal and malignant single cells from 13 tumor types across 17 different studies. All of the above studies may be used to generate pseudobulk cohorts to test how purity affects a classifier's prediction as seen in FIG. 3A. Filtered, quality controlled expression data and metadata for the cells used in the study may be downloaded from the curated cancer cell atlas, 3CA website (https://www.weizmann.ac.il/sites/3CA/). Cancer single-cell sequencing data may be processed with several steps. For example, from 17 studies representing 13 different tumor types the following sub-cohorts may be created:
1) For each of 205 patients representing 13 different tumor types, one may aggregate weighted sum counts of each normal and malignant cell population together (see following section, In-silico generation of tumor-normal mixed sample cohort, for details). This cohort may be used to test the machine learning classifier accuracy at various tumor purity levels.
2) 10 of the most abundant non-malignant cell types may be selected from the single cell sample cohort. For each patient that had at least one of the selected cell types, the expression counts of all cells of the same cell type may be summed together to create a per cell type, per patient, pseudobulk expression profile for a total of 860 samples. This cohort may be used to test the overall capability of the developmental deconvolution to separate different normal cell types as seen in FIG. 5B.
3) From a random cohort of 10 patients (two of each of the following tumor types BRCA, COADREAD, LUNG, OV and PAAD), 100 random T-cells and 100 random fibroblasts may be chosen from each, forming a total of 2000 individual unique single cells (1000 T Cells and 1000 Fibroblasts). These may then be used to test the quality of the developmental deconvolution on isolated cells as shown in FIG. 4B and FIG. 5A.
4) All the malignant and non-malignant cells from two LIHC samples may be individually correlated with the MOCA dataset to test the effect of the normal and malignant cell compartments on developmental component profiles of an aggregated sample as shown in FIG. 4C.

FIGS. 5A-5B show an example of developmental deconvolution radar plots for eight randomly chosen T-cells and normal cell types from scRNAseq. FIG. 5A: 8 T-cells were chosen at random from those sequenced in scRNAseq studies analyzed and their radar plots are shown for comparison to one another. FIG. 5B: For each of the listed cell types, the annotated normal cells of that type across scRNAseq studies were aggregated by summing counts, performing deconvolution, and plotting on the radar (see Methods). Note that other normal (non-malignant) cell types may be present in some samples in some studies, the cell types above were chosen for analysis due to their consistent annotation across all studies.

### In-Silico Generation of a Tumor-Normal Mixed Sample Cohort

In-silico generation of a tumor-normal mixed sample cohort may be part of data processing. To test the effect of tumor purity on classifier accuracy (FIGS. 3A-3B), one may take two approaches: 1) mixing of matched primary tumor and normal bulk TCGA samples and 2) pseudobulk of tumor and normal mixing of single cells of known malignant or non-malignant type from the same sample. For approach 1, the TCGA contains 678 primary tumor samples for which normal matched samples are available, across 17 different tissues: BLCA, BRCA, CESC, CHOL, COADREAD, HNSC, KIDNEY, LIHC, LUNG, PAAD, PCPG, PRAD, SARC, STES, THCA, THYM, UCEC. For every primary tumor with matched normal sample the gene expression profiles may be processed to create a gradient of mixed gene expression ranging from 100% tumor samples to 100% normal tissue with 10% increments (90% tumor+10% normal, 80% tumor+20% normal etc.). For approach 2, all the counts from malignant and non-malignant cells from the same patient were summed to create a malignant pseudobulk and a normal pseudobulk. The expression profiles of the pseudobulked normal and malignant compartment were then mixed with an analogous strategy to that used for TCGA tumor-normal patient samples, testing purity content ranges from 100% malignant only pseudobulk, to 100% non-malignant expression profile with 10% increments. These in silico generated samples were correlated with MOCA cells, deconvoluted and inputted in the D-MLP classifier. The result of this analysis is shown in FIGS. 3A-3B.

### Example Organ Development Datasets

It was described above that the single-cell atlas 120 may, for example, include gene expression data representing single cells derived from mammal samples in developmental stages, or may include data representing normal development for each of a plurality of mammalian development trajectories. The single-cell atlas could be, for example, the Mouse Organogenesis Cell Atlas (MOCA), and/or the Human Fetal Organs (HFO) dataset.

In brief, the Mouse Organogenesis Cell Atlas (MOCA) contains single cell RNA sequencing data for organogenesis in mice. MOCA arranges single cells into developmental trajectories, and contains RNA sequencing data for 1,331,984 cells. MOCA defines developmental trajectories; the process of defining developmental trajectories is described in the paper introducing the MOCA dataset. The Human Fetal Organs (HFO) dataset contains single cell RNA sequencing data of 377,456 single cells from 15 human fetal organs. Below, more detail is provided about MOCA and HFO.

### The Mouse Organogenesis Cell Atlas (MOCA)

The expression profile and meta information of MOCA cells RNA sequencing may be manually downloaded from: https://oncoscape.v3.sttrcancer.org/atlas.gs.washington.edu.mous e.rna/downloads. The expression data of the 1,331,984 high quality cells defined in the MOCA study may be used. Filtering may be applied to MOCA data. MOCA filtering may include: cells with less than 400 detected mRNA molecules were removed, all detected doublet cells removed, and all cells from doublet derived sub-clusters were removed. In the MOCA study, the authors identified 10 main and 56 sub, non-continuous trajectories based on transcriptional similarities between the analyzed cells and literature curated marker genes. These trajectories may be used as part of calculation of biological component scores.

### Human Fetal Organs (HFO) Single Cell Dataset

In another example, the single-cell atlas 120 may include the Human Fetal Organs (HFO) single cell dataset. The HFO dataset includes expression data of 377,456 single cells from 15 human fetal organs and relative meta data which may be downloaded from https://descartes.brotmanbaty.org/bbi/human-gene-expression-during-development/.

Similarities between TCGA correlations with MOCA and HFO may be analyzed. In order to test the specificity and reproducibility of the correlation between the mouse expression profiles and human expression profiles one may adopt the following approach. TCGA expression profiles may be correlated with HFO cells as previously described. The HFO cell types may then be mapped to the MOCA sub trajectories. The correlation coefficient between the two similarity matrices (each 56×62, see formulas) may then be calculated by cor.test( )R implementation. For per TCGA sample type correlations, the same function may be applied across the appropriate column of the matrices.

### High-Level Overview of the Diagnosis of Malignancy by Developmental Deconvolution and Machine Learning

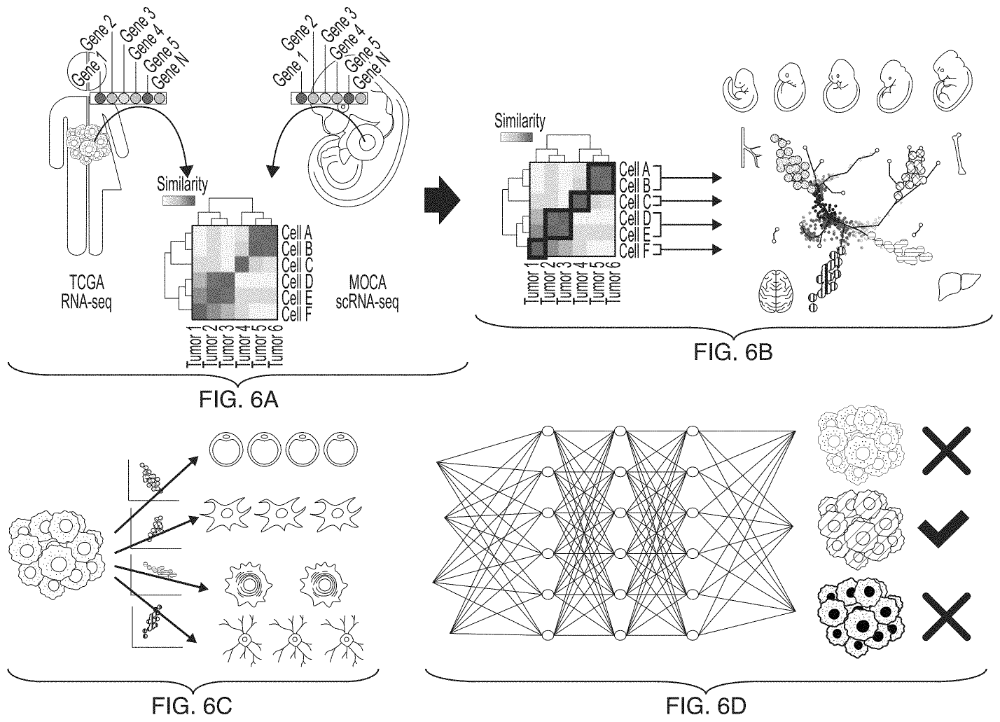

FIGS. 6A-6D provide a high-level example of diagnosis of malignancy by developmental deconvolution and machine learning. FIG. 6A: A mapping between bulk RNA-sequencing data from The Cancer Genome Atlas (TCGA) (an example of the tumor atlas 118) and single cell RNA-sequencing data from the Mouse Organogenesis Cell Atlas (MOCA) (an example of a single-cell atlas 120) was performed, to yield an example of the map 116. FIG. 6B: Each mapped relationship consists of tumor types and developmental sub-trajectories, represented at different stages of embryogenesis. FIG. 6C: Using this map, bulk gene expression signatures from each tumor sample can be deconvoluted into component developmental trajectories. FIG. 6D: Scores for each developmental trajectory at each embryonic timepoint (an example of biological component scores 124) can be inputted into a multilayer perceptron classifier (an example of a classifier 114) that outputs tumor type prediction. This classifier is then applied to cancer of unknown primary.

### Overview of the Mapping Process

It was described above that a map generation module 132 may generate a map 116 that maps from (some or all of) the gene expression data for the plurality of training tumors in the tumor atlas 118 to (some or all of) the gene expression data representing single cells in the single-cell atlas 120. It was further described above that such generation of the map 116 may be performed, for example, using normalization and rank-based, non-parametric approaches. In brief, the mapping process used by the map generation module 132 may include mouse-human gene name conversion followed by similarity score calculation and processing. For example, similarity score calculation may first involve calculation of Spearman's rank-ordered correlation coefficient of MOCA single-cell RNA sequencing data versus TCGA bulk-sequenced cancer samples. Spearman's correlation coefficient is calculated on the RNA sequence data of shared genes. This yields a matrix of dimension (number of cells in MOCA study) by (number of samples in TCGA). Next, the similarity score matrix is further processed. Each column is mean centered and standard deviation scaled. For each MOCA single cell, the Spearman correlation coefficients from the same TCGA tumor sample type are averaged. Then, an average is taken across all cells of the same MOCA developmental sub-trajectory to arrive at a single similarity score relating each tumor type to each developmental sub-trajectory. For MOCA and TCGA, the matrix produced by this step consists of 56 MOCA developmental sub-trajectories by 62 TCGA tumor types. Finally this matrix is processed with column-wise mean centering and standard deviation scaling, and min-max normalization, to yield a final similarity scores matrix that relates tumor types to developmental trajectories. This final similarity scores matrix is an example of the map 116 and the aforementioned steps are an example of the mapping process that a map generation module 132 may perform. Note that the steps for mapping can be performed between any organ development sample (e.g. MOCA and/or HFO), and any tumor sample (e.g. TCGA, non-TCGA, and/or MGH samples).

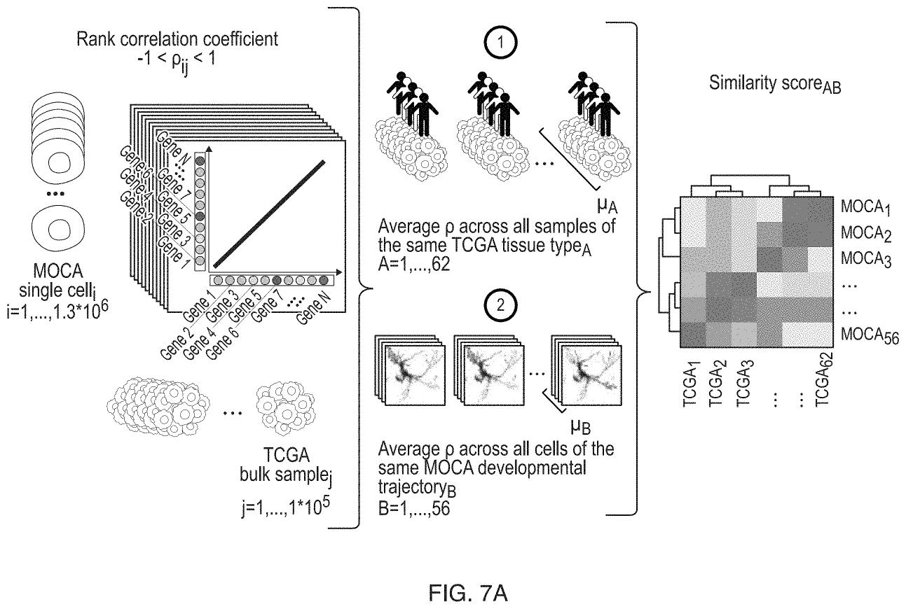

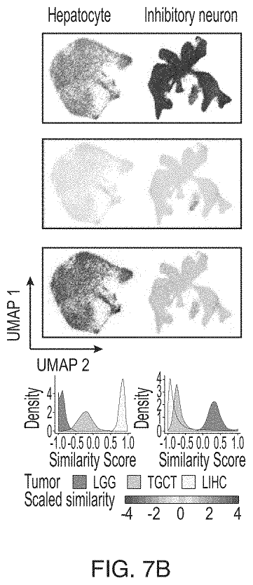

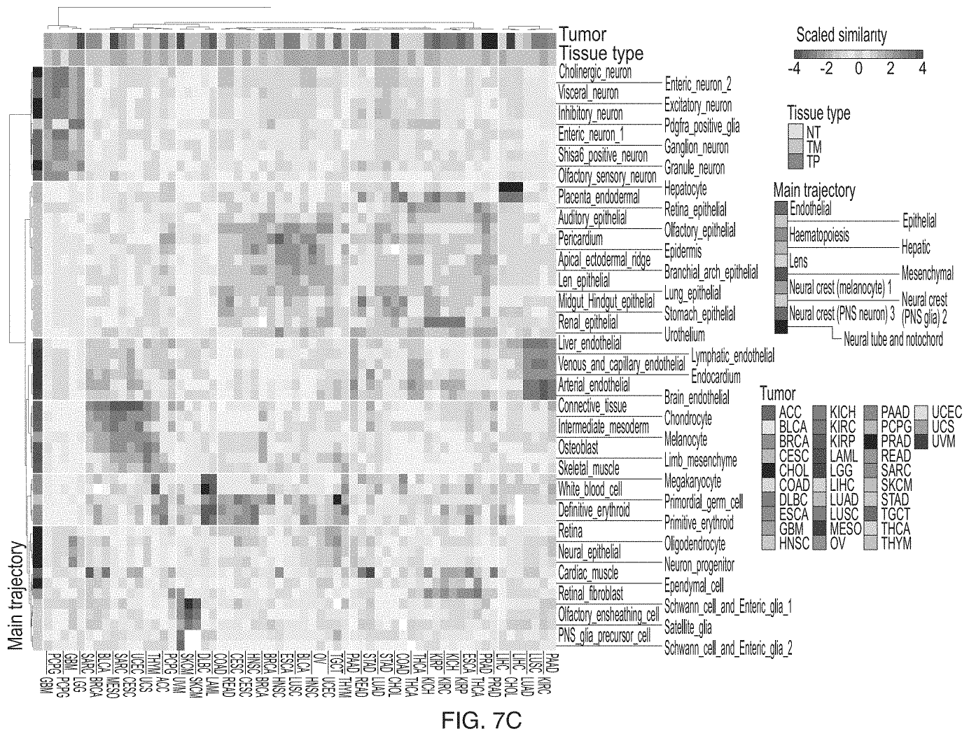

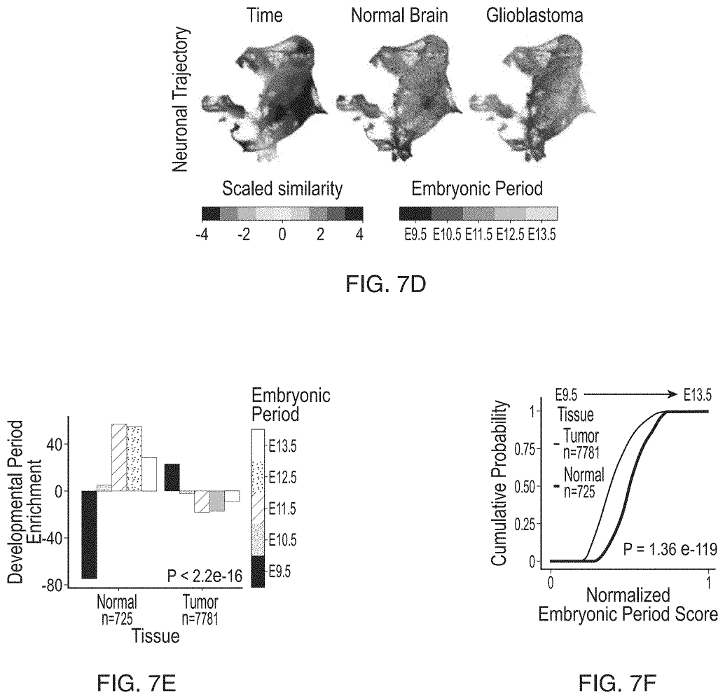

An example of the mapping process used by the map generation module 132 is shown in FIGS. 6A and 6B, which illustrate mapping of tumors to trajectories belonging to major cell lineages and developmental programs through systematic comparison of gene expression profiles of TCGA samples with MOCA single cells. FIG. 7A illustrates more specifically a part of an example mapping process in the rank-based correlation coefficient is calculated for expressed genes between each TCGA sample and each MOCA single cell. Altogether, this example analysis constitutes systematic comparison between 15,929 genes expressed across 10,388 TCGA samples derived from 9,681 patients and 1,331,984 single cells derived from the MOCA dataset. In total, 21,217,199,458 data points may be used to compute 13,836,649,792 correlation. The following sections provide additional methodologic details about the mapping process: “Mouse-Human Gene Names Conversion,” “Similarity Score Calculation,” and “Similarity score aggregation, scaling and normalization.”

### Mouse-Human Gene Names Conversion

The first step of the mapping process may be mouse-human gene names conversion. For example, to compare murine gene expression from MOCA to human gene expression in TCGA, gene names may be standardized between mouse and human using the following approach. A conversion from Mouse Ensembl id (MOCA) to Human gene symbol/Entrez id (TCGA) may be achieved using BioMart (https://www.ensembl.org/biomart/martview/) Ensembl v95. Human gene symbol/Entrez id (TCGA) to Ensembl id (NON-TCGA) mapping may be achieved using org.Hs.egENSEMBL2EG from org.Hs.eg.db (v3.8.2) Bioconductor (v3.9) (https://bioconductor.org/) package. The intersection of these two sources may be performed using the Human gene symbol/Entrez id shared identifier. This process may generate a list of translated names. This list may then be used as a dictionary for gene names and mouse-human orthologue comparison. In an example, this may identify on the order of 15,929 unique human genes that may be used. In the case of multiple mouse gene names mapping to the same human gene name, the average expression levels may be calculated across occurrences.

### Similarity Score Calculation

The second step of the mapping process may be similarity score calculation. The similarity between gene expression profiles from either MOCA or human fetal organs (HFO) cells and bulk (TCGA, NON-TCGA, MGH samples) or single cells from cancer datasets may be calculated by means of Spearman's Correlation Coefficient, implemented using cor function in R on the expression profile of all shared genes identified as described above for each bulk/single cell sample and MOCA cell. Spearman's Correlation Coefficient may provide advantages because this non-parametric, rank-based approach is more robust to outliers caused by single cell transcript dropout and is unaffected by the normalization method, which standardized the use of different gene expression datasets.

Spearman correlation generates a matrix C of correlation coefficients of dimensions n×m, where:
n represents the number of individual cells from the organ development study, e.g. MOCA or HFO. For the MOCA study, n=1,331,984. For HFO, n=377,456.
m represents the number of samples from the comparing tumor study, e.g. TCGA or MGH. For TCGA, m=10,393 (9274 primary tumors, 394 metastasis and 725 normal tissues). For MGH, m=85.
   The matrix of correlation coefficients for MOCA/TCGA samples is termed C_{MOCA/TCGA}, and is a 1331984×10393 matrix, containing 1.38e+10 correlation coefficients. C_{HFO/TCGA} is a 377456×10393 matrix containing 3.92e+09 correlation coefficients, and C_{MOCA/MGH} is a 1331984×85 matrix. The correlation coefficient may then be used as a metric for the similarity between the samples in the cohorts under exam. Correlation coefficients may then be further processed as described in the next section, “Similarity score aggregation, scaling and normalization.”

### Similarity Score Aggregation, Scaling and Normalization

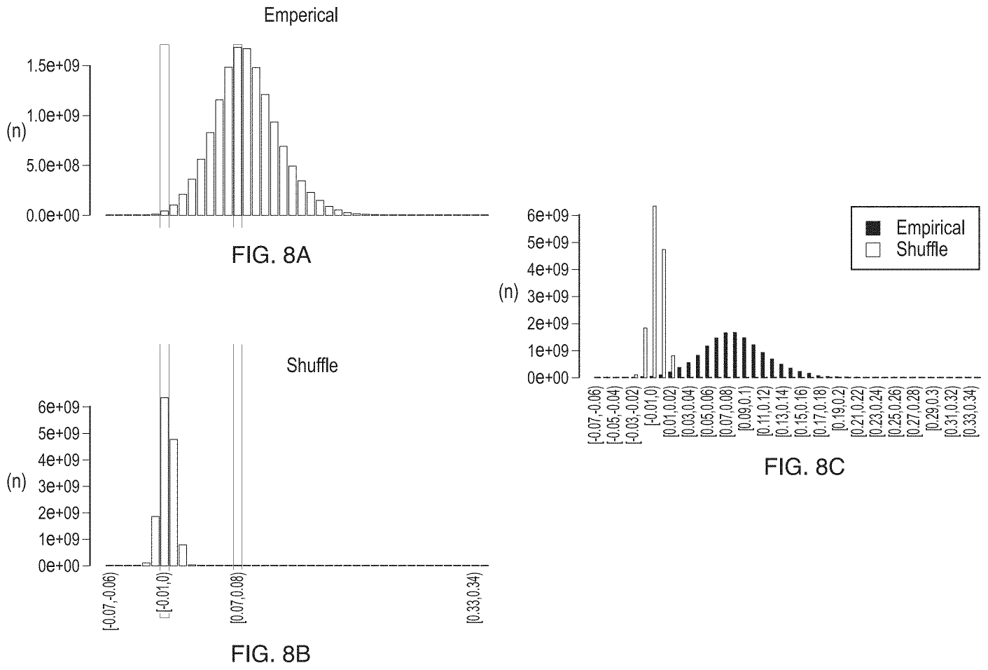

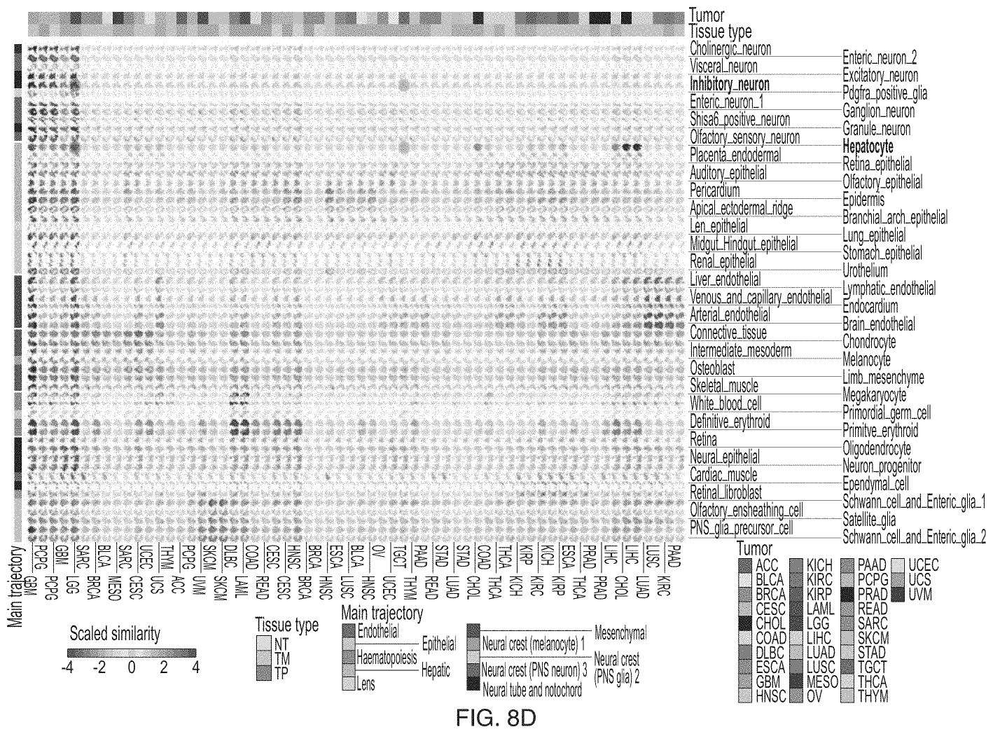

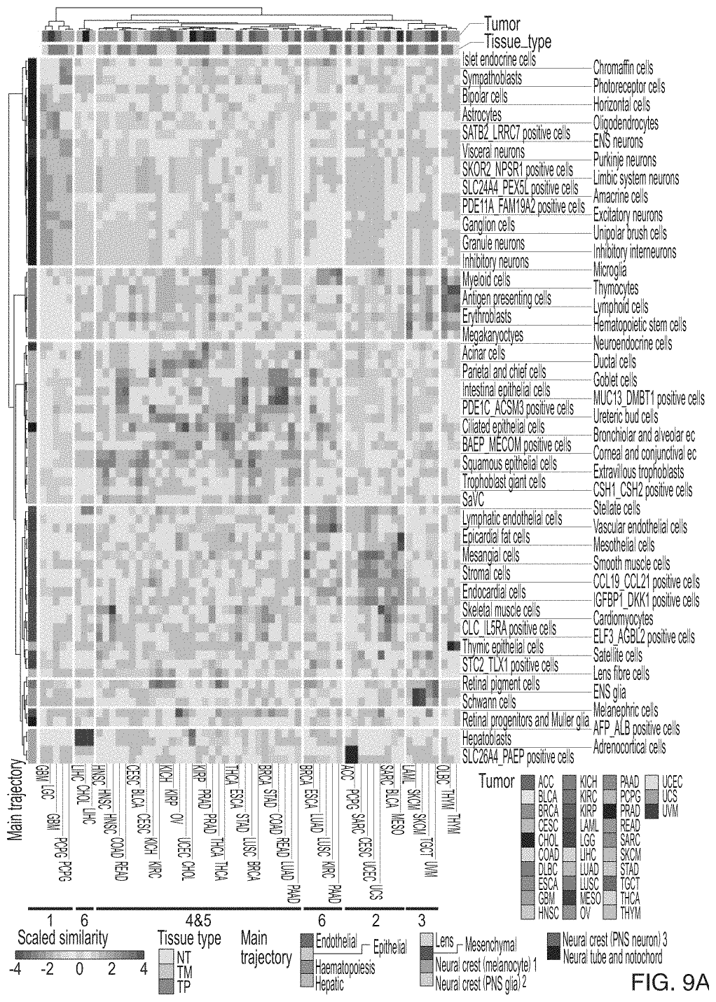

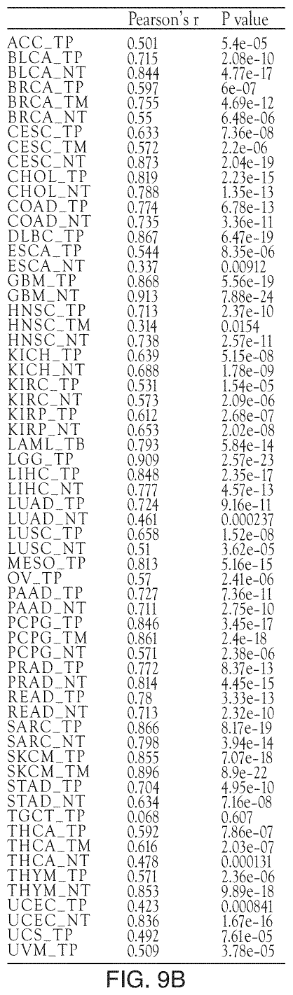

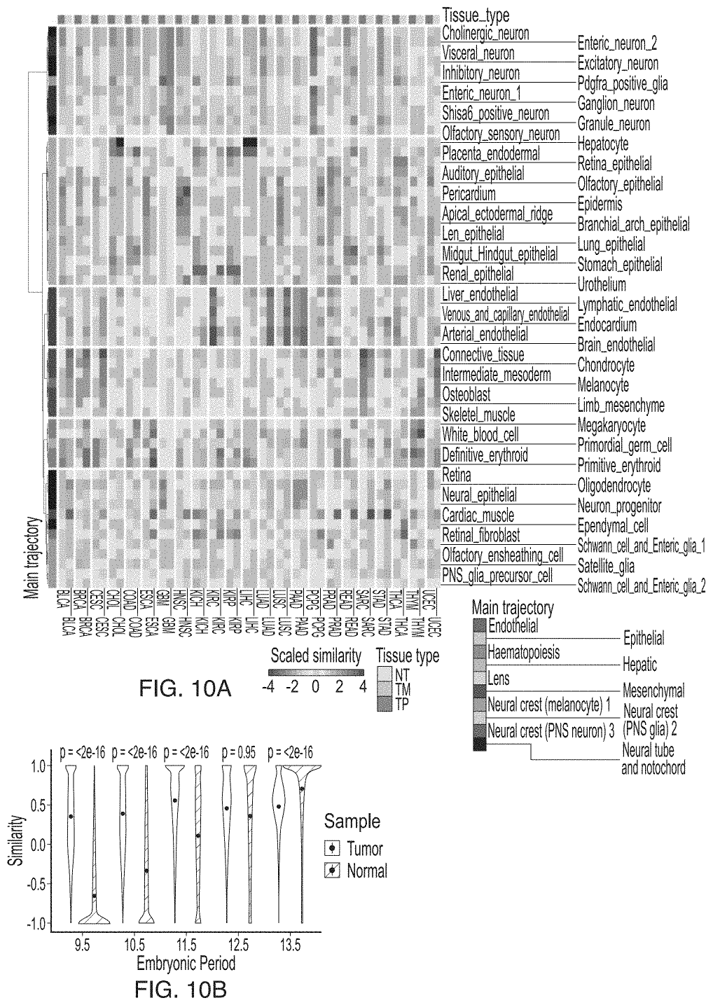

To generate TCGA aggregated similarity scores shown in FIG. 7B, FIG. 7C, FIG. 8D, FIG. 9A, FIG. 9B, and FIG. 10A, dimensions of matrices C_{MOCA/TCGAA}, C_{HFO/TCGA} and C_{MOCA/MGH} the following steps may be performed.
Step 1: Begin with the n×m matrix of correlation coefficients described above, wheren is the number of cells in the organ development study (MOCA or HFO), and m is the number of samples in the comparing tumor study (TCGA or MGH).
Step 2: Each column of matrix C is mean centered and standard deviation (σ) scaled resulting in matrix C̅ calculated using scale( )R function.
Step 3: The scaled values for every tumor dataset sample belonging to the same tissue type are then averaged. k is the number of unique tumor dataset sample types (TCGA tissue types) so this step yields matrix Ĉ′ with dimensions n×k. For TCGA, k=62, meaning the new matrix has the same number of rows (n) but a reduced number of columns (k).
Step 4: Every cell belonging to the same sub-trajectory is then averaged which yields the matrix Ĉ″. l indicates the total number of unique sub-trajectories, so the matrix Ĉ″ has dimensions l×k. Ĉ″ for MOCA and TCGA samples now consists of 56 sub-trajectories×62 tissue type scores (derived from 33 tumor types for which a combination of primary, metastatic and normal tissues are available).
Step 5: A column-wise mean centered, and standard deviation scaled version of Ĉ″ is calculated as in scaled similarity scores.
Step 6: Scaled values are further min-max normalized to change the range of the scaled similarity scores to plotted interval (e.g. (0−1), or (−1, +1) as shown in the figures.
   Overall, after these aggregation, scaling, and normalization steps, a final matrix of similarity scores is produced. This final matrix of similarity scores is an example of a map 116 that provides a mapping between the tumor atlas 118 and the single-cell atlas 120.

FIGS. 7A-7F provide a visualization of the systematic correlation of TCGA to developmental trajectories. FIG. 7A: The MOCA developmental atlas contains n=1,331,984 high confidence scRNAseq cells, and for each the gene expression was systematically correlated (Spearman's rank-ordered correlation coefficient, ρ) against each of the m=10,388 samples from TCGA. Correlation coefficients were scaled across the dataset by Z-score. In step 1, for each cell, the correlation coefficients from the same TCGA sample type were averaged and plotted; k=62 is the number of unique sample types. Then, in step 2, for each MOCA developmental trajectory these correlation coefficients were further averaged to arrive at a single similarity score relating each tumor type to each developmental trajectory, depicted below (FIG. 7C); l=56 is the number of unique developmental trajectories. Sample types are defined in the TCGA study and developmental trajectories in the MOCA study and were used as given. FIG. 7B: Cells from the MOCA sub-trajectories hepatocyte development and inhibitory neuron development are plotted according to their UMAP coordinates given in that study. Similarity of each cell with selected TCGA sample types is shown, as is the distribution of similarity scores across the cells for each TCGA type (LGG=low grade glioma, TGCT=testicular germ cell tumor, LIHC=liver hepatocellular carcinoma). Inhibitory neuron trajectory cells showed higher similarity scores with LGG than do hepatocyte trajectory cells, and vice versa for LIHC. TGCT was not significantly related to either trajectory. FIG. 7C: Heatmap showing the scaled similarity between every developmental sub-trajectory and each TCGA sample type. TCGA samples from normal tissue (NT, green), metastatic tumors (TM, gray), and primary tumors (TP, aqua) are indicated at top. Main developmental trajectory types are defined by the MOCA dataset for each sub-trajectory and shown at left. Sub-trajectory names are listed at right. Hierarchical clustering of rows (trajectories) and columns (sample types) is shown. FIG. 7D: Neuronal progenitor cells are plotted according to their UMAP coordinates and colored according to their embryonic timepoint of isolation (left), similarity to normal brain samples (middle) or glioblastoma samples (right). Quantification in FIG. S5B. FIG. 7E: Pan-cancer comparison of tumor samples with normal samples for enrichment of the embryonic days from which their most similar developmental cells are derived. Chi-square testing p-value is shown. FIG. 7F: Cumulative distribution (CDF) plot of normalized embryonic period score for all tumor and normal samples in TCGA is shown. Kolmogorov-Smirnov testing p-value is shown. TCGA code names: ACC=Adrenocortical carcinoma, BLCA=Bladder Urothelial Carcinoma, LGG=Brain Lower Grade Glioma, BRCA=Breast invasive carcinoma, CESC=Cervical squamous cell carcinoma and endocervical adenocarcinoma, CHOL=Cholangiocarcinoma, COAD=Colon adenocarcinoma, ESCA=Esophageal carcinoma, GBM=Glioblastoma multiforme, HNSC=Head and Neck squamous cell carcinoma, KICH=Kidney Chromophobe, KIRC=Kidney renal clear cell carcinoma, KIRP=Kidney renal papillary cell carcinoma, LAML=Acute Myeloid Leukemia, LIHC=Liver hepatocellular carcinoma, LUAD=Lung adenocarcinoma, LUSC=Lung squamous cell carcinoma, MESO=Mesothelioma, OV=Ovarian serous cystadenocarcinoma, PAAD=Pancreatic adenocarcinoma, PCPG=Pheochromocytoma and Paraganglioma, PRAD=Prostate adenocarcinoma, READ=Rectum adenocarcinoma, SARC=Sarcoma, SKCM=Skin Cutaneous Melanoma, STAD=Stomach adenocarcinoma, TGCT=Testicular Germ Cell Tumors, THYM=Thymoma, THCA=Thyroid carcinoma, UCS=Uterine Carcinosarcoma, UCEC=Uterine Corpus Endometrial Carcinoma, UVM=Uveal Melanoma.

FIGS. 8A-8D illustrate example developmental correlations between TCGA and the MOCA dataset. FIG. 8A: The distribution of the 13,836,649,792 correlation coefficients calculated by taking the Spearman correlation coefficient for gene expression between each cell in the developmental atlas (MOCA) and each sample in the tumor atlas (TCGA). To compare gene expression across species, a standardized set of orthologous genes was used and expression of species specific genes was ignored. FIG. 8B: Gene rows were shuffled in each MOCA cells such that the same counts per cell were used but assigned to a random gene, and then were correlated against each TCGA sample as in A. The distribution of correlation coefficients is shown. FIG. 8C: Overlay of histograms in A and B. FIG. 8D: Each column contains 1,331,984 high confidence scRNAseq cells from the developmental atlas colored by similarity to each sample type in TCGA. The cells are grouped by sub-trajectory as defined by the MOCA study (each row is a sub-trajectory, and each column a TCGA sample type) and plotted according to their UMAP coordinates given by the MOCA dataset. Similarity was measured using Spearman's rank-ordered correlation coefficient (ρ) for expression of each gene between each cell and each TCGA sample, scaled across the dataset by Z-score (‘Scaled Similarity’, blue to red), and coefficients averaged for each TCGA sample type (see also FIG. 7A). Main trajectories, sub-trajectories and TCGA sample types are as in FIG. 7C. The sub-trajectories highlighted in FIG. 7B are background shaded accordingly to allow their identification here.

Verifying that Calculated Coefficients Represent Meaningful Associations

It is possible to verify that the calculated coefficients in the map 116, represented in an example by the similarity scores matrix, display meaningful association between the two datasets by comparing them to those generated from row-randomized data (FIGS. 8A-8C). In the MOCA study, single cells are grouped into 10 main trajectories, which are then divided into 56 sub-trajectories, using gene expression similarity and known marker genes. Further, for each sub-trajectory, MOCA provides Uniform Manifold Approximation (UMAP) coordinates for all the cells of that trajectory. One may average correlation coefficients for each sample of the same TCGA type (FIG. 7A) and plot their similarity against cells in each MOCA sub-trajectory (FIG. 8D). In an example, this revealed many expected relationships, such as inhibitory neuronal trajectories showing similarity with low grade gliomas (LGG) but not hepatocellular tumors (LIHC), and vice versa for hepatocyte trajectories (FIG. 7B).

In the next part of this analysis, one may average correlation coefficients across all cells of the same developmental sub-trajectory (FIG. 7A), and visualize them as a single similarity score compared against TCGA sample type (FIG. 7C). In an example, hierarchical clustering analysis of these data identified 6 TCGA sample and 6 developmental sub-trajectory clusters, for a total of 36 (FIG. 7C). Clusters highlighted relationships between tissue types and developmental trajectories: brain derived samples (LGG, GBM) with neuronal sub-trajectories; kidney tumors (KICH, KIRP) with renal epithelial trajectories; hepatocellular tumors (LIHC) with hepatocyte trajectories; and testis tumors (TCGT) with germ cell trajectories, amongst several other expected correlations. Furthermore, we observed expected developmental lineage relationships: melanoma samples (SKCM) with neural crest trajectories, carcinoma tumors with epithelial lineages, and mesenchymal tumor types with mesoderm derived developmental lineages all showed strong similarity (FIG. 7C). Identification of expected relationships supported the notion that the observed correlations were due to underlying biological relationships and served as partial validation of the method.

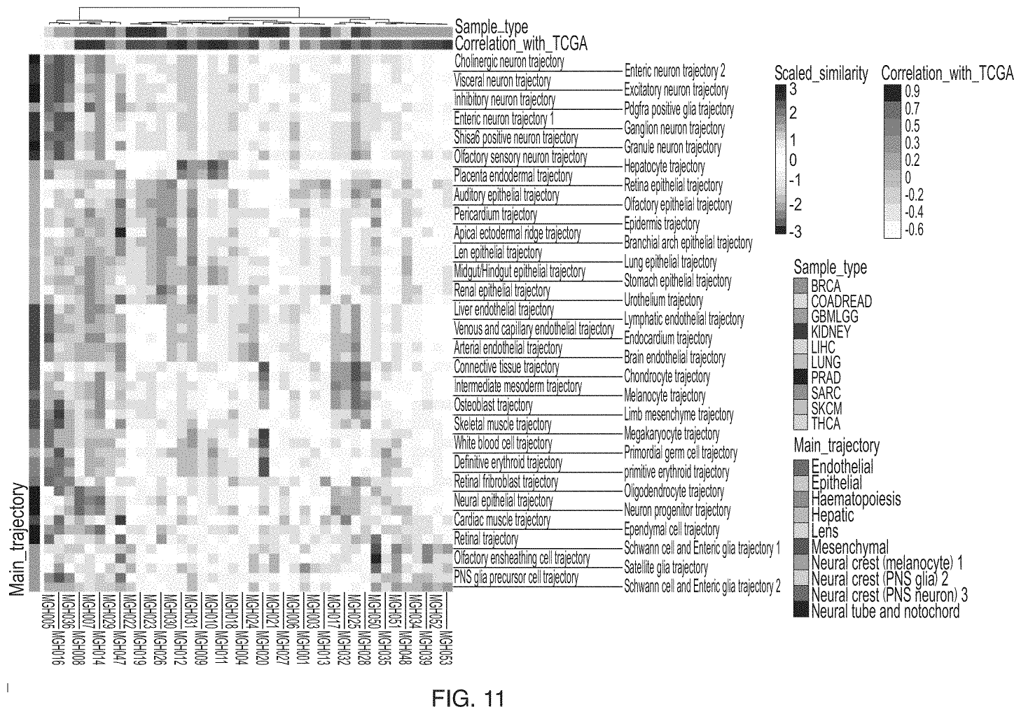

In order to further validate the observed correlations, we employed two approaches. In the first approach, we developed an optimized protocol for transcriptome sequencing from formalin fixed paraffin embedded tissue (FFPE), sequenced the transcriptome for 40 tumors of known types, and compared similarity for developmental trajectories to TCGA. Comparison between the FFPE cohort and TCGA cohort showed strong agreement, validating the method in an independent sample set. FIG. 11 illustrates the known sample type (histopathological diagnosis, top row) and the Spearman correlation coefficient (second row) for the rank order of trajectory correlation for each MGH FFPE sample (columns) against the equivalent TOGA primary tumor type. The average Spearman correlation coefficient across all multiple hypothesis corrected correlations is 0.69. Note, two samples (far left) appear anti-correlated when comparing FFPE correlated trajectories to TOGA correlated trajectories; removal of these two from the analysis raises the value to 0.76.

In the second approach, we utilized a single cell atlas of human fetal tissues cataloguing later embryonic stages of mid-gestation development. More specifically, during the course of our study a partial atlas of mid-gestation human fetal development became available, and we used a representative set of cells provided by the human atlas, correlated them with TOGA sample types, and compared the results to those from the murine atlas. FIGS. 9A-9B provide an example of validation of TOGA and MOCA correlation using human developmental trajectories. FIG. 9A: We correlated the human developmental atlas with TOGA samples, collapsed coefficients using the same technique as was done for the mouse developmental atlas (FIGS. 7A-7F), and plotted similarity for each human developmental cell type (rows) against each TOGA sample type (columns), ordered by hierarchical clustering. At bottom, numbers indicate the analogous cluster on the TOGA-MOCA heatmap (FIG. 7C), numbered left to right. Human cell types in the atlas are defined in existing literature. A remarkably similar set of clusters was obtained when compared to the TOGA-MOCA correlation heatmap (FIG. 7C). FIG. 9B: For each TOGA tumor type, the correlation between mouse (MOCA) and orthologous human fetal developmental trajectory values is shown by Pearson correlation coefficient. Orthologous trajectories are defined in the human fetal atlas developmental study. Note that this is a correlation of correlations across individual patient samples for each TCGA type. The overall Pearson R coefficient across all TCGA types and all orthologous trajectories is R=0.78.

### Pan-Cancer Comparisons of Tumor-Normal Tissues and Embryonic Period

Pan-cancer comparisons of tumor-normal tissues and embryonic period may be performed. For every TCGA sample from tissue types that contained the expression profile of at least one normal sample, the number of the most strongly correlated cells (top 1,331 cells, sorted by correlation coefficient), was binned by their embryonic period of origin (E9.5-E13.5). This created a matrix of either normal or malignant samples versus the 5 embryonic time periods (E9.5, E10.5, E11.5, E12.5 and E13.5), wherein each matrix entry indicates the number of MOCA cells in the given category. This matrix was then analyzed using the chi-squared test to produce FIG. 7E. The developmental period enrichment represents the test residuals calculated as (observed-expected)/sqrt(expected). For FIG. 7F the actual embryonic day (represented as an integer, 9.5, 10.5 and so on) was first multiplied by the number of cells in its relative column, then these values were added together, providing a per sample measure characterizing the development period of its transcriptional profile.

### Deconvolution By Developmental Components (DCs)

It was described above that the system 110 may include a biological component score generation module 122 that generates a biological component score representing the training tumor as a compilation of non-gene terms describing known biological programs (such as a plurality of known developmental programs).

In an example, the creation of a correlation map between TCGA samples and developmental trajectories inspired us to attempt a systematic developmental deconvolution of human tumor gene expression. In deconvolution, a recorded signal (bulk gene expression) made of component parts (developmental programs) is deconstructed into individual signals from each component (trajectories at embryonic timepoints). We used developmental components (DC), a single quantitative measure of each developmental sub-trajectory at each timepoint, to represent the developmental information for every TCGA sample. DCs were scaled across all tumor samples and charted on radar plots, which represent information about developmental period, sub-trajectory, and DC score for each sample. A schematic for this plot is shown (FIG. 4A). A deconvolution score was generated for each developmental sub-trajectory at each embryonic timepoint for a total of 214 scores (developmental components); the 214 developmental component scores are the inputs to the developmental multilayer perceptron.

To perform the deconvolution into developmental components, the following steps may be performed: For each TCGA, NON-TCGA, MGH bulk and single cell sample the MOCA cells were sorted in increasing (lowest to highest) order based on their correlation coefficient. Next, the 1,331 most strongly correlated cells were selected, representing the top ˜0.1% of all MOCA cells tested. A rank-based score was then assigned to this selection of cells. The most highly correlated cell was given a score of 1331, while the least correlated score was given a score of 1. These scores were then summed across all cells belonging to the same combination of sub-trajectory at a particular developmental time, creating the raw developmental component (DCs) score. The raw scores were then transformed by taking the natural logarithm (ln). The correlation between the DCs and sets of samples was calculated using the Kruskal-Wallis rank sum test using the kruskal.test( )R function. Only DCs with a Benjamini-Hochberg adjusted p-value<0.05 were considered statistically different between groups.

FIGS. 4A-4D provide an example of developmental deconvolution capturing signals from normal and malignant cells. FIG. 4A: Illustration of radar plots for depicting results of developmental deconvolution. Multiple layers of information are present. For each sample, a deconvolution score is generated for each developmental sub-trajectory (#3) at each embryonic timepoint (#1) for a total of 214 scores (developmental components, or ‘DCs’). Scores are represented as the distance from center in the innermost circle. The main trajectory (#2) of each sub-trajectory is also indicated and colored, as is the relative number of samples showing each signal within each radar plot or FIG. 4A (#4, purple to yellow). FIG. 4B: Results of developmental deconvolution performed on one T-Cell or fibroblast cell from scRNAseq (left) or the aggregated signal from 1,000 single cells (right). FIG. 4C: For one liver hepatocellular tumor (LIHC) sequenced by scRNAseq in study [ref], the signals for all aggregated normal (non-malignant) and malignant cells are shown. FIG. 4D: Developmental deconvolution of one bulk-sequenced liver hepatocellular tumor is shown for comparison.

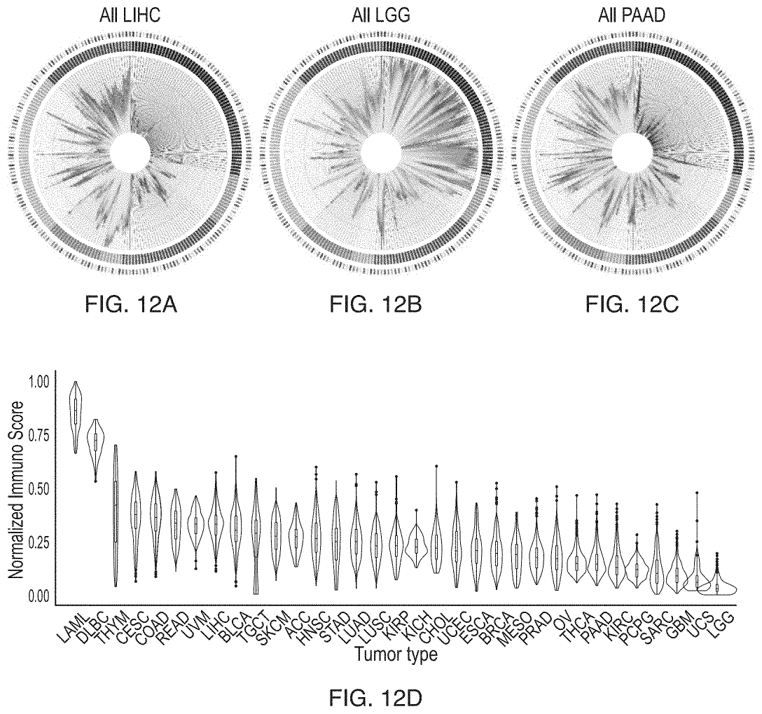

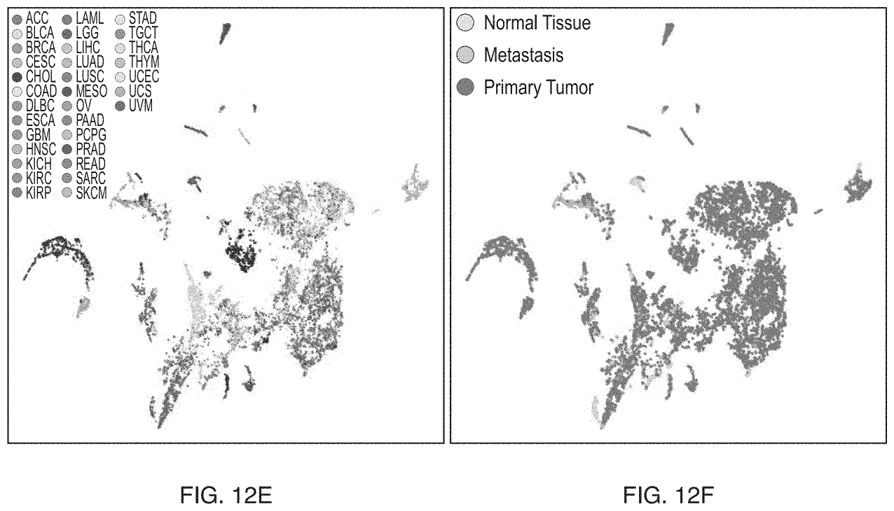

FIGS. 12A-12F provide an example of developmental deconvolution of tumor samples. FIG. 12A: Radar plot showing the developmental deconvolution signals for all TOGA hepatocellular carcinoma samples hepatocellular carcinoma samples. FIG. 12B: Radar plot showing trajectory deconvolution signals for all low-grade glioma samples. FIG. 12C: Radar plot showing trajectory deconvolution signals for all pancreatic adenocarcinoma (PAAD) samples. FIG. 12D: An immune infiltrate score (‘immuno score’) was calculated for each TOGA tumor sample as the sum of deconvolution scores for relevant trajectories. The distribution of scores for each TOGA tumor type is shown (box plot center median, box edges 25^{th} to 75^{th} percentiles). FIG. 12E: UMAP dimensionality reduction was performed on the 214 developmental deconvolution scores for each TOGA sample and plotted. Tumor type is indicated. See also FIGS. 3A-3B for each tumor type colored separately. FIG. 12F: Identical to E but each sample is colored by sample type (normal, primary tumor, metastasis).

FIGS. 10A-10B provide an example of tumor-normal tissue type comparison for developmental trajectories. FIG. 10A: Shown is a heatmap equivalent to FIG. 7C, arranged by placing each primary tumor adjacent to the equivalent normal tissue for all patient sample types for which matched normal data was available in TOGA. Main and sub-trajectories and scaled similarity scores are as in FIG. 7C. FIG. 10B: Violin plots showing the distribution of similarity scores for each sample type (glioblastoma, normal brain) at each embryonic timepoint (E9.5-E13.5) as shown in FIG. 7D, dots indicate the median cell. P-values for Mann-Whitney U-tests at each timepoint are shown.

### Developmental Multilayer Perceptron

It was described above that the machine learning classifier 114 may be capable of outputting a tumor type of an input tumor based on gene expression data for the input tumor. The machine learning classifier 114 may be implemented as a multilayer perceptron (MLP). An MLP is a type of supervised machine learning model. An MLP trained on natural log transformed and min-max normalized developmental component (DC) scores may be termed a developmental multilayer perceptron (D-MLP). As an example, the D-MLP may have 214 deconvolution scores/developmental component scores (DC scores) as input; these DC scores are calculated as the similarity of each tumor's gene expression to embryologic developmental trajectories. The D-MLP may output scores for the 27 aggregated TCGA tumor classes. Examples used for training and testing the D-MLP may come from the TCGA, non-TCGA, and-or institution-specific (MGH cohort) datasets. To ensure reproducibility, the minimum and maximum values of the aggregated TCGA, non-TCGA and MGH cohort were calculated and used as the minimum and maximum for all min-max normalization, including for the normalization of the CUP cohort. The model's hyper-parameters were identified by means of grid search over the following variables: 1) number of hidden layers, 2) the number of nodes per layer, 3) the type of optimizer function, 4) the type of loss indicator and 5) the number of epochs for which the model is trained. The architecture selection and the training/validation of the model were performed by a 10-fold cross validation of a training-validation set split of 60%-10% of the totality of the TCGA, NON-TCGA and MGH cohorts. The performance of the D-MLP was tested on the remaining test set (30% of the whole cohort). None of the samples present in the test set were used during the model training or during the architecture selection. Further, the performance of the D-MLP was tested on an independent cohort of single cell derived-pseudo bulk samples. The final model has the following architecture: 1 input layer with 214 nodes, 2 hidden layers of 800 and 200 nodes, respectively and 1 output layer with 27 nodes. The model was trained with a stochastic gradient descent optimizer using a mean squared error loss function. Accuracy was calculated as a performance metric. The model was trained on the training set for 300 epochs with an early stopping function based on the accuracy score, with a patience of 3 epochs. The D-MLP was implemented in Python (v3.6.4) using sklearn (v0.19.1) and Keras (v2.2.0, with TensorFlow backend).

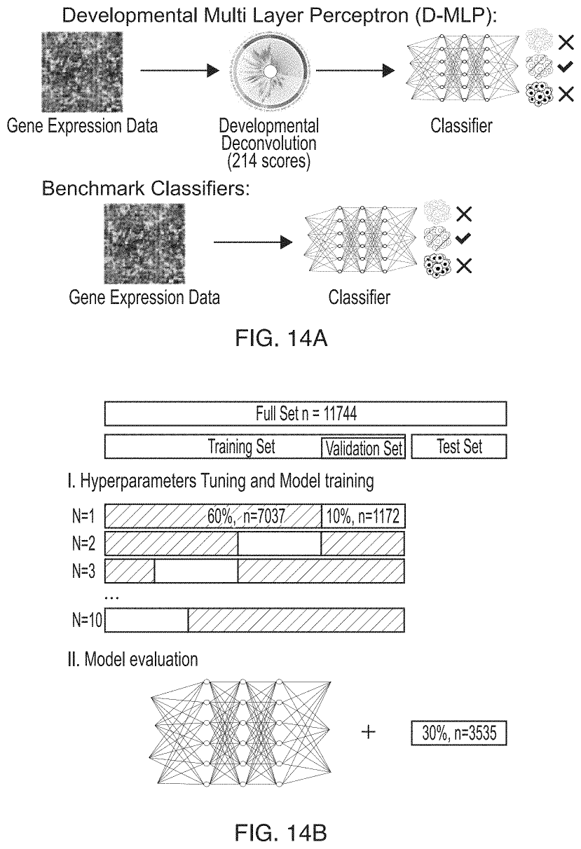

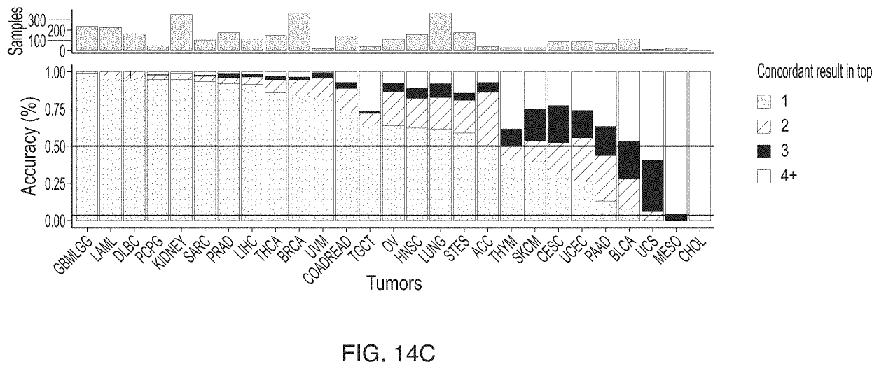

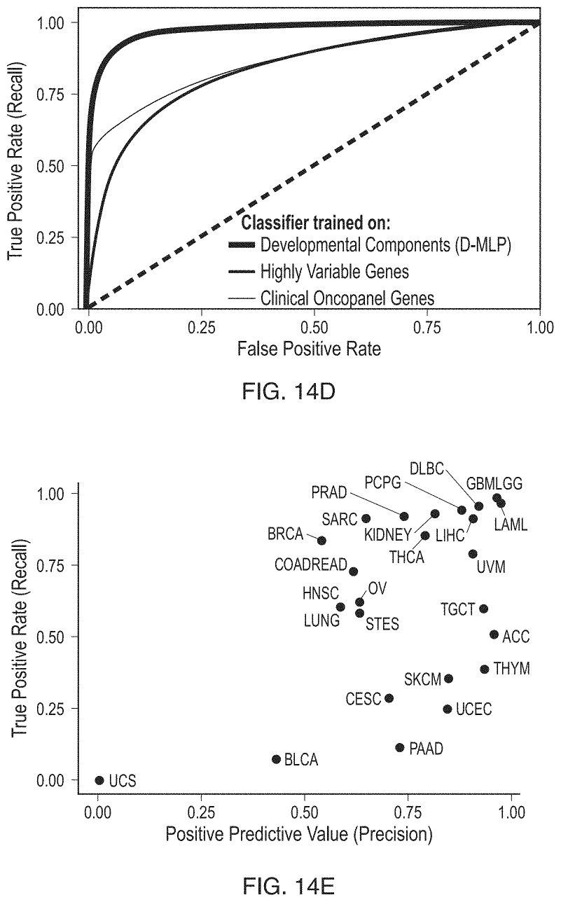

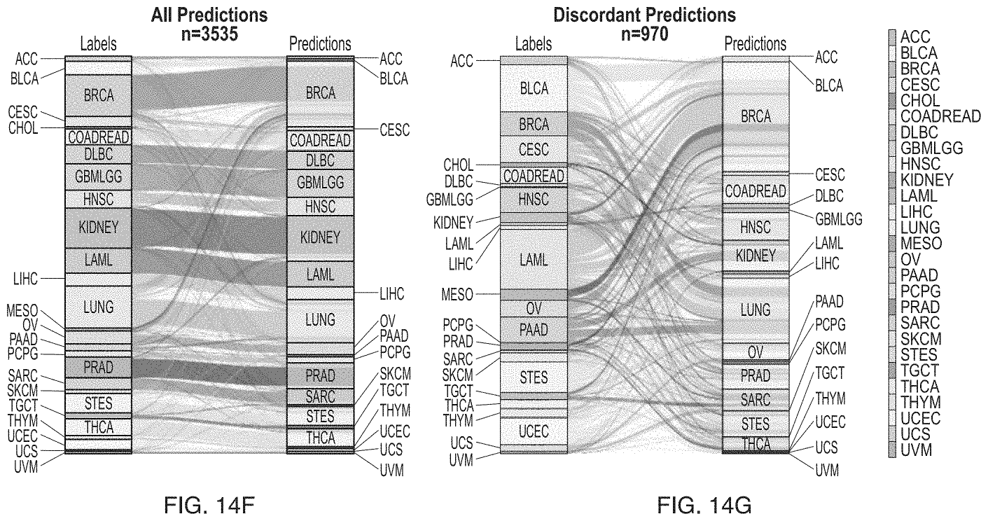

FIGS. 14A-14G illustrate an example of construction and testing of a developmental multilayer perceptron (D-MLP) classifier for tumor type. FIG. 14A: Schematic for classifier construction and testing. The D-MLP classifier uses developmental deconvolution scores calculated as the similarity of each tumor's gene expression to embryologic developmental trajectories (FIG. 7 through FIG. 12) as input. Comparison is made against benchmark classifiers that use gene expression data directly. FIG. 14B: Parameter optimization and model training. The full cohort contains samples from multiple cancer studies (TCGA, BEATAML1.0, CGCI-BLGSP, CTSP-DLBCL1, MMRF-COMPASS and TARGET). Of the full cohort (11,744 samples), (i) 70% of cases were sampled, 60% for training and 10% for validation, in hyper-parameter optimization using a 10-fold cross validation approach to construct the classifier. (ii) 30% of cases were held out and never seen by the model during training or optimization (Test set). The model was assessed on these cases to gauge performance. FIG. 14C: Classifier accuracy (concordance) measured against the test set and number of samples are shown for all TCGA tumor types. FIG. 14D: Micro-averaged receiver operator curve (ROC) plotting the true positive rate (also known as recall) as a function of false positive rate for classifier performance for the top prediction. D-MLP classifier performance (blue line) and random guess performance (dashed grey line) are shown. Area under the curve (ROC-AUC) for the top prediction was calculated as 0.974+/−0.003. Also shown are ROC performance curves for benchmark classifiers trained on either the most highly variable genes in expression across the training cohort (‘highly variable genes’, light grey, ROC-AUC: 0.859 (95% confidence interval 0.829−0.976) or expression of a panel of genes tested in routine clinical assays at our institution (‘clinical oncopanel genes’, dark grey, ROC-AUC: 0.836 (95% confidence interval 0.828−0.975). FIG. 14E: Precision (positive predictive value) versus recall (true positive rate) performance characteristics for D-MLP for each TCGA tumor type. Note CHOL and MESO are omitted as the positive predictive value is undefined for these tumors. FIG. 14F: Sankey plot showing classifier results for the top tumor type prediction for all samples (n=3,535). FIG. 14G: Sankey plot showing the results for discordant classifications (n=1,006) for top predictions.

### D-MLP Classification Analysis

A classification analysis may be performed of the results of the D-MLP. In an example, the raw likelihood score resulting from the classification of the test set, CUP cohort, tumor purity cohort, and benchmark cohorts were each analyzed as follows. The output of the D-MLP classifier is a matrix containing a number of rows equal to the number of samples analyzed and a number of columns equal to 27, for the 27 classification labels. Each sample's top classification (defined by highest likelihood score) is assigned as top1, the next as top2, and so on (up to 4+ as shown in FIGS. 5A-5B and S12). For each tumor type, a frequency table is generated of the number of top1/top2/top3/4+ predictions over the number of samples analyzed. To compare the output to the true tumor labels, one-hot encoding is used to create another matrix where each entry is 1 if the given sample is labeled with the given tumor label in the original dataset, and 0 otherwise. The output classification matrix together with the encoded matrix are then used to calculate true positive rate, false positive rate, and ROC-AUC. The confidence interval for the ROC-AUC scores were calculated over 1000 bootstraps. The results of classification of the various cohorts are shown in FIGS. 14A-14G, FIGS. 13A-13C, and FIGS. 3A-3B.

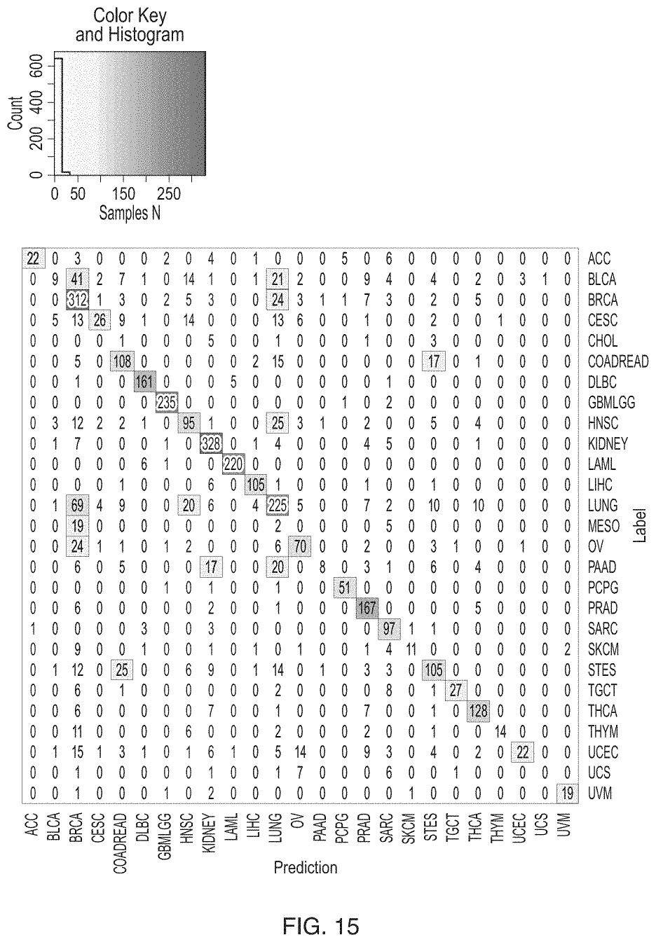

FIG. 15 shows a confusion matrix for an example D-MLP's performance. The confusion matrix includes all the top1 prediction results for the example D-MLP classifier. The y-axis represents the sample tumor type as assigned by the study from which it was obtained, and x-axis the tumor type labels for the prediction of the classifier. Diagonal results represent true positives. Note the matrix is not perfectly square as CHOL and MESO are omitted from columns as the example classifier never predicted these outcomes (0 columns).

FIGS. 3A-3B demonstrate an example of validation of a D-MLP classifier using scRNAseq and tumor-normal mixing. FIG. 3A: 13 tumor types from scRNAseq tumor studies were analyzed using an example D-MLP. For each tumor type, all cells annotated in the study classified as non-malignant (normal, consisting of tumor associated stroma and infiltrating cells) and malignant cells were analyzed per patient, with the number of patients analyzed for each tumor type indicated. To simulate 100% tumor purity, for each patient gene expression for all malignant cells was combined, analyzed by deconvolution, and inputted into D-MLP. The resulting classification accuracy was analyzed as shown above by accuracy and ROC. To simulate samples of varying tumor purity, gene expression was combined for randomly chosen malignant and non-malignant cells in the indicated ratios (e.g. 0.7 means 7 malignant cells for every 3 normal cells), deconvolution performed, and analyzed by D-MLP for classification accuracy. The overall accuracy across all tumor types is shown at top, along with ROC curves and ROC-AUC analysis for each ratio of malignant cells to normal cells. FIG. 3B: For the cohort of bulk-sequenced samples analyzed by D-MLP in the study (FIG. 5B), we simulated tumor-normal mixes for samples where matched patient normal bulk-sequencing samples were available and annotated tumor purity was >90%. Using the bulk-tumor sequencing as the malignant cells and matched patient normal as the non-malignant cells, we simulated mixed tumor samples by weighting gene expression between the two using the indicated ratios. For example, at 0.7, the expression of each gene in the tumor sample was multiplied by 0.7 and in the normal sample by 0.3 and added together for the mix. Gene expression was then deconvoluted into DCs and inputted into D-MLP, with accuracy and ROC results shown. Note that the accuracy is higher than shown in FIGS. 14A-14G due to the fact that several tumor types with low accuracy of prediction did not have available matched normal samples, and were thus omitted from this analysis.

Grouping of discordant predictions may be used as part of a performance assessment of a D-MLP. As an example, to assess the distance between top3 classification results of discordantly classified samples and concordantly classified samples, the following ‘hot-encoder’ approach was taken. Each sample's classification response was encoded using a vector of length 81 (27 possible prediction labels with a spot for each of the top3 predictions). Each element of this vector was then populated with a score of 5, 3, 2 or 0 depending whether the correct answer was in the top1, top2, top3, or top4 (all other predictions) category. A Top1 (correctly classified tumor) would have a score of 5 repeated 3 times, a top2 correctly classified sample would have a score of 3 in the range 28 to 54, and so on. For example, an ACC sample correctly classified in top1 would have a score of 5 in position 1, 28 and 55 (and 0s everywhere else) while an ACC sample guessed right in top2 would have a score of 3 in position 28 and 0s everywhere else. These vectors were then compared using cosine similarity , and UMAP was used to plot the results of cosine similarity analysis.

CUPs clustering and DC analysis may be performed. Raw DC scores of the CUP cohort were processed as follows. Each DC score was mean centered and standard deviation scaled (z-scored) across the whole cohort of 52 CUPs. After scaling, Spearman distance was calculated between different samples. This distance was evaluated by means of Dist( ) function in the amap R package. The total number of samples is x; the function outputs an x×x matrix with the pairwise distance between x samples. A hierarchical clustering analysis using ‘ward.D’ algorithm was then calculated on this distance matrix using hclust( ) R function and cut into 4 main clusters (chosen based on observed distance between branch points) using cutree( ) R function. Statistical analysis of the differential developmental programs between the 4 clusters was performed by the Kruskal-Wallis test using kruskal.test( ) in R. Enrichment for specific classifications was performed using the chi-squared test. 70 DCs with a Bonferroni corrected p-value<0.05 were considered correlated with at least one cluster and are shown in FIG. S15A.

The D-MLP may be compared to alternative configurations of machine learning models. In an example, the performance of the D-MLP classifier was tested against models sharing the same architecture trained on pure gene expression profiles directly without developmental deconvolution. This analysis included two sets of genes: I) Clinical oncopanel genes and II) highly variable genes. I) Clinical oncopanel genes represent a list of 251 genes tested in routine clinical cancer care at the Massachusetts General Hospital (assays: SNaPshot, Solid Fusion Assay, Heme SNaPshot). To match feature counts with D-MLP classifier, we generated 10 random subsets of 214 genes out of 251. Each of these 214 gene subsets were used to train a benchmark classifier, and the highest performing assessed by top1 and top3 accuracy was directly compared against the developmental based classifier (D-MLP) as shown (FIG. 14C). For the highly variable gene benchmark classifier, the 214 most variably expressed genes assessed by pure variance (using the var( ) function in R) across the full pan-cancer cohort (TCGA, NON-TCGA and MGH) taken altogether was used. Expression of these genes in TPM was used as input for the benchmark classifier.

### Diagnosis of Cancer of Unknown Primary by a Developmental Multilayer Perceptron Classifier

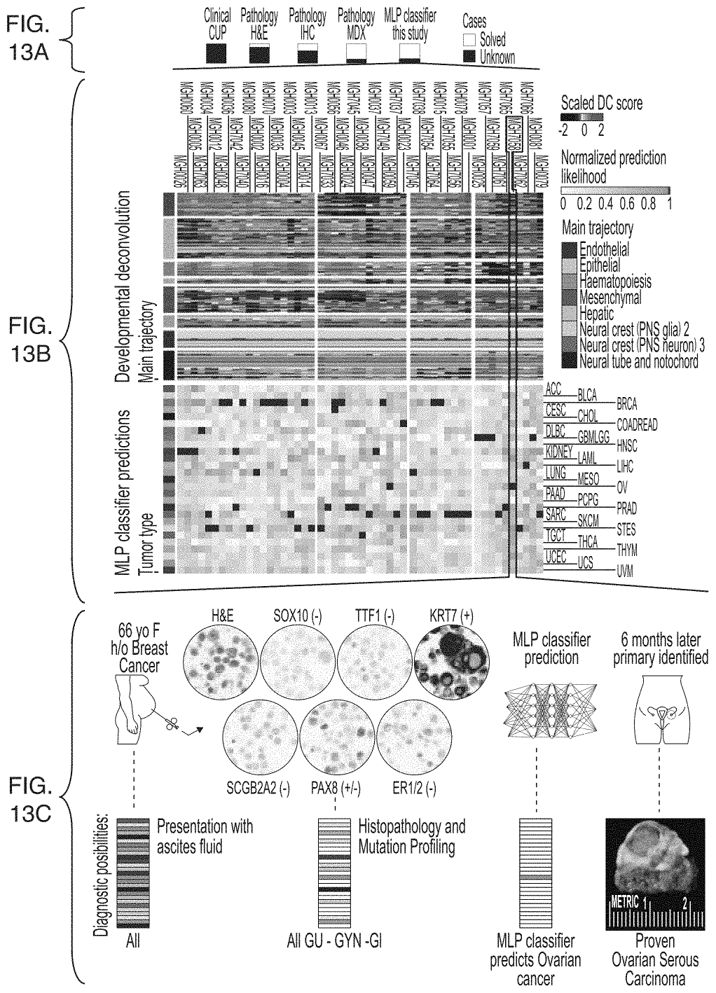

As described above, the classifier 114 may be applied to gene expression data for an input tumor (e.g., the input tumor data 104). This input tumor may be a Cancer of Unknown Primary (CUP). FIGS. 13A-13C illustrate an example of diagnosis of Cancer of Unknown Primary by a developmental multilayer perceptron classifier. FIG. 13A: Patients present clinically with CUP. Some cases are solved by hematoxylin and eosin (H&E) examination of tissue. Additional cases are solved by immunohistochemistry stains (IHC) that mark particular tissue types and by molecular techniques (MDX) such as mutation profiling. However, a subset of CUP remains undiagnosed with no primary site assigned after all available techniques. We applied D-MLP a cohort of 52 such cases from our institution. FIG. 13B: (Top) The developmental deconvolution profile of each CUP case is shown by plotting developmental component scores. Trajectories are arranged top to bottom as they are shown counter-clockwise on radar plots (FIGS. 7A-7F), and colors for main trajectories are as in FIGS. 7A-7F) through FIGS. 14A-14G. (Bottom) As described above, the input tumor may not be among the plurality of training tumors; i.e., the classifier 114 can generate tumor types for tumors that were not in the tumor atlas 118 that was used to construct the classifier 114. The (Bottom) of this figure shows D-MLP classifier predictions for 52 CUP cases. Note higher confidence predictions are weighted in coloration (dark green). Colors for tumor types are as in FIG. 7 through FIG. 14. FIG. 13C: Case MGH058 is highlighted for further consideration. A 66-year-old female with a history of breast cancer presented with ascites fluid accumulation. Fluid was drained and assessed by standard cytological/histopathological workup. Stains are shown for H&E (morphology), mammaglobin A (SCG2A2, breast cancer), estrogen receptor (ER1/2, breast cancer), TTF1 (lung), SOX10 (melanoma), Cytokeratin 7 (KRT7, epithelial origin), and PAX8 (broad marker, primarily genitourinary), which ruled out breast cancer but left a broad differential diagnosis encompassing genitourinary (GU), gynecological (GYN), and some gastrointestinal (GI) malignancies. D-MLP classifier gave a strong prediction of ovarian cancer for this patient's ascites. 6 months later, after extensive workup, the patient underwent bilateral salpingo-oophorectomy and was found to have a mass (pictured) identified as ovarian serous carcinoma.

### Statistical Analysis

Statistical analysis may be performed using R (v3.6.3). Enrichment (FIG. 7E) may be calculated using a chi-squared test and represented as (observed-expected)/ sqrt(expected). Statistical differences between cumulative distributions may be evaluated using Kolmogorov-Smirnov (K-S) tests, e.g. as in FIG. 7F. Pairwise differences between means of continuous variables from different samples may be evaluated using Mann-Whitney tests as in FIG. 10B. Receiver-operator characteristics may be calculated using roc curve and roc auc functions from Python3 sklearn (v0.22.1) using 1,000 bootstraps to calculate empirical 95% confidence intervals. Non-parametric approaches used include Spearman's correlation coefficient, the chi-square test, the Kruskal-Wallis rank sum test, the Kolmogorov-Smirnov test, and the Mann-Whitney test.

One embodiment of the present invention is directed to a method, performed by at least one computer processor executing computer program instructions stored on at least one non-transitory computer-readable medium, for using a map which maps from gene expression data for a plurality of training tumors in a tumor atlas to gene expression data representing single cells derived from mammal samples in developmental stages in a single-cell atlas. The method includes: (A) using the map to extract, from the plurality of training tumors, a plurality of biological components, thereby generating, for each of the plurality of training tumors and each of the plurality of biological components, a corresponding biological component score representing the training tumor as a compilation of non-gene terms describing a plurality of biological programs; and (B) constructing, based on the map, a machine learning perceptron classifier that outputs a tumor type of an input tumor based on gene expression data for the input tumor, wherein the input tumor is not among the plurality of training tumors.

The method may further include: (C) before (A), generating the map. Generating the map may include mapping each of the plurality of training tumors to at least one of the plurality of mammalian developmental trajectories in the single-cell atlas. Operation (C) may include: (C)(1) receiving the single-cell atlas; (C)(2) receiving the tumor atlas; and (C)(3) generating the map based on the single-cell atlas and the tumor atlas. The gene expression data for the plurality of training tumors may include, for each of the plurality of training tumors: (1) gene sequencing data for the training tumor; and (2) a label indicating a type of cancer of the training tumor. The gene expression data representing single cells derived from mammal samples in developmental stages in the single-cell atlas may include gene expression data representing organogenesis of a plurality of mammalian developmental trajectories.

The tumor atlas may include a version of The Cancer Genome Atlas (TOGA).

The single-cell atlas may include a developmental atlas. The single-cell atlas may include a single-cell organogenesis atlas. The single-cell atlas may include data representing normal development for each of a plurality of mammalian developmental trajectories. The single-cell atlas may include a version of the Mouse Organogenesis Cell Atlas (MOCA).

The plurality of known biological programs may include a plurality of known developmental programs.

The method may further include: (C) applying the machine learning perceptron classifier to the gene expression data for the input tumor to generate the tumor type of the input tumor. The input tumor may include a sample of a cancer of unknown primary.

Using the map to extract, from the plurality of training tumors, the plurality of biological components may include using the map to deconvolute the plurality of training tumors into the plurality of biological components.

The developmental stages in the single-cell atlas may include prenatal developmental stages in the single-cell atlas.

Another embodiment of the present invention is directed to a method performed by at least one computer processor executing computer program instructions stored on at least one non-transitory computer-readable medium. The method includes: (A) receiving a single-cell atlas comprising gene expression data; (B) receiving a tumor atlas comprising gene expression data for a plurality of training tumors; and (C) generating a map, which maps from the gene expression data in the tumor atlas to the gene expression data in the single-cell atlas.

The gene expression data for the plurality of training tumors may include, for each of the plurality of training tumors: (1) gene sequencing data for the training tumor; and (2) a label indicating a type of cancer of the training tumor. The operation (C) may include generating the map using normalization and rank-based, non-parametric approaches.

The gene expression data in the single-cell atlas may include data representing organogenesis of a plurality of mammalian development trajectories. Generating the map may include mapping each of the plurality of training tumors to at least one of a plurality of mammalian developmental trajectories in the single-cell atlas. The gene expression data for the plurality of training tumors may include, for each of the plurality of training tumors: (1) gene sequencing data for the training tumor; and (2) a label indicating a type of cancer of the training tumor.

Another embodiment of the present invention is directed to a machine learning perceptron classifier generated by a method. The machine learning perceptron classifier is stored in at least one non-transitory computer-readable medium. The method includes: (A) using a map to extract, from a plurality of training tumors in a tumor atlas, a plurality of biological components, thereby generating, for each of the plurality of training tumors and each of the plurality of biological components, a corresponding biological component score representing the training tumor as a compilation of non-gene terms describing a plurality of biological programs, wherein the map maps from gene expression data for the plurality of training tumors in the tumor atlas to gene expression data representing single cells derived from mammal samples in developmental stages in a single-cell atlas; and (B) constructing, based on the map, the machine learning perceptron classifier, wherein the machine learning perceptron classifier is adapted to output a tumor type of an input tumor based on gene expression data for the input tumor, wherein the input tumor is not among the plurality of training tumors.

The gene expression data for the plurality of training tumors may include, for each of the plurality of training tumors: (1) gene sequencing data for the training tumor; and (2) a label indicating a type of cancer of the training tumor. The gene expression data representing single cells derived from mammal samples in developmental stages in the single-cell atlas may include gene expression data representing organogenesis of a plurality of mammalian developmental trajectories. The tumor atlas may include a version of The Cancer Genome Atlas (TOGA).

The single-cell atlas may include a developmental atlas. The single-cell atlas may include a single-cell organogenesis atlas. The single-cell atlas may include data representing normal development for each of a plurality of mammalian developmental trajectories. The single-cell atlas may include a version of the Mouse Organogenesis Cell Atlas (MOCA).

The plurality of known biological programs may include a plurality of known developmental programs. The input tumor may include a sample of a cancer of unknown primary.

Using the map to extract, from the plurality of training tumors, the plurality of biological components may include using the map to deconvolute the plurality of training tumors into the plurality of biological components. The developmental stages in the single-cell atlas may include prenatal developmental stages in the single-cell atlas.

It is to be understood that although the invention has been described above in terms of particular embodiments, the foregoing embodiments are provided as illustrative only, and do not limit or define the scope of the invention. Various other embodiments, including but not limited to the following, are also within the scope of the claims. For example, elements and components described herein may be further divided into additional components or joined together to form fewer components for performing the same functions.

Any of the functions disclosed herein may be implemented using means for performing those functions. Such means include, but are not limited to, any of the components disclosed herein, such as the computer-related components described below.

The techniques described above may be implemented, for example, in hardware, one or more computer programs tangibly stored on one or more computer-readable media, firmware, or any combination thereof. The techniques described above may be implemented in one or more computer programs executing on (or executable by) a programmable computer including any combination of any number of the following: a processor, a storage medium readable and/or writable by the processor (including, for example, volatile and non-volatile memory and/or storage elements), an input device, and an output device. Program code may be applied to input entered using the input device to perform the functions described and to generate output using the output device.

Embodiments of the present invention include features which are only possible and/or feasible to implement with the use of one or more computers, computer processors, and/or other elements of a computer system. Such features are either impossible or impractical to implement mentally and/or manually. For example, embodiments of the present invention using machine learning to train a model to generate diagnoses of tumors. Such training, and subsequent use, of a machine learning model is inherently rooted in computer technology and could not otherwise be performed mentally or manually by a human.

Any claims herein which affirmatively require a computer, a processor, a memory, or similar computer-related elements, are intended to require such elements, and should not be interpreted as if such elements are not present in or required by such claims. Such claims are not intended, and should not be interpreted, to cover methods and/or systems which lack the recited computer-related elements. For example, any method claim herein which recites that the claimed method is performed by a computer, a processor, a memory, and/or similar computer-related element, is intended to, and should only be interpreted to, encompass methods which are performed by the recited computer-related element(s). Such a method claim should not be interpreted, for example, to encompass a method that is performed mentally or by hand (e.g., using pencil and paper). Similarly, any product claim herein which recites that the claimed product includes a computer, a processor, a memory, and/or similar computer-related element, is intended to, and should only be interpreted to, encompass products which include the recited computer-related element(s). Such a product claim should not be interpreted, for example, to encompass a product that does not include the recited computer-related element(s).

Each computer program within the scope of the claims below may be implemented in any programming language, such as assembly language, machine language, a high-level procedural programming language, or an object-oriented programming language. The programming language may, for example, be a compiled or interpreted programming language.

Each such computer program may be implemented in a computer program product tangibly embodied in a machine-readable storage device for execution by a computer processor. Method steps of the invention may be performed by one or more computer processors executing a program tangibly embodied on a computer-readable medium to perform functions of the invention by operating on input and generating output. Suitable processors include, by way of example, both general and special purpose microprocessors. Generally, the processor receives (reads) instructions and data from a memory (such as a read-only memory and/or a random access memory) and writes (stores) instructions and data to the memory. Storage devices suitable for tangibly embodying computer program instructions and data include, for example, all forms of non-volatile memory, such as semiconductor memory devices, including EPROM, EEPROM, and flash memory devices; magnetic disks such as internal hard disks and removable disks; magneto-optical disks; and CD-ROMs. Any of the foregoing may be supplemented by, or incorporated in, specially-designed ASICs (application-specific integrated circuits) or FPGAs (Field-Programmable Gate Arrays). A computer can generally also receive (read) programs and data from, and write (store) programs and data to, a non-transitory computer-readable storage medium such as an internal disk (not shown) or a removable disk. These elements will also be found in a conventional desktop or workstation computer as well as other computers suitable for executing computer programs implementing the methods described herein, which may be used in conjunction with any digital print engine or marking engine, display monitor, or other raster output device capable of producing color or gray scale pixels on paper, film, display screen, or other output medium.

Any data disclosed herein may be implemented, for example, in one or more data structures tangibly stored on a non-transitory computer-readable medium. Embodiments of the invention may store such data in such data structure(s) and read such data from such data structure(s).

Any step or act disclosed herein as being performed, or capable of being performed, by a computer or other machine, may be performed automatically by a computer or other machine, whether or not explicitly disclosed as such herein. A step or act that is performed automatically is performed solely by a computer or other machine, without human intervention. A step or act that is performed automatically may, for example, operate solely on inputs received from a computer or other machine, and not from a human. A step or act that is performed automatically may, for example, be initiated by a signal received from a computer or other machine, and not from a human. A step or act that is performed automatically may, for example, provide output to a computer or other machine, and not to a human.

The terms “A or B,” “at least one of A or/and B,” “at least one of A and B,” “at least one of A or B,” or “one or more of A or/and B” used in the various embodiments of the present disclosure include any and all combinations of words enumerated with it. For example, “A or B,” “at least one of A and B” or “at least one of A or B” may mean: (1) including at least one A, (2) including at least one B, (3) including either A or B, or (4) including both at least one A and at least one B.

What is claimed is:

1. A method, performed by at least one computer processor executing computer program instructions stored on at least one non-transitory computer-readable medium, for using a map which maps from gene expression data for a plurality of training tumors in a tumor atlas to gene expression data representing single cells derived from mammal samples in developmental stages in a single-cell atlas, the method comprising:
(A) using the map to extract, from the plurality of training tumors, a plurality of biological components, thereby generating, for each of the plurality of training tumors and each of the plurality of biological components, a corresponding biological component score representing the training tumor as a compilation of non-gene terms describing a plurality of biological programs; and
(B) constructing, based on the map, a machine learning perceptron classifier that outputs a tumor type of an input tumor based on gene expression data for the input tumor, wherein the input tumor is not among the plurality of training tumors.

2. The method of claim 1, further comprising:
(C) before A, generating the map.

3. The method of claim 2, wherein generating the map comprises mapping each of the plurality of training tumors to at least one of the plurality of mammalian developmental trajectories in the single-cell atlas.

4. The method of claim 2, wherein (C) comprises:
(C) (1) receiving the single-cell atlas; and
(C) (2) receiving the tumor atlas;
(C) (3) generating the map based on the single-cell atlas and the tumor atlas.

5. The method of claim 4, wherein the gene expression data for the plurality of training tumors comprises, for each of the plurality of training tumors: (1) gene sequencing data for the training tumor; and (2) a label indicating a type of cancer of the training tumor.

6. The method of claim 4, wherein the gene expression data representing single cells derived from mammal samples in developmental stages in the single-cell atlas comprises gene expression data representing organogenesis of a plurality of mammalian developmental trajectories.

7. The method of claim 1, wherein the tumor atlas comprises a version of The Cancer Genome Atlas (TOGA).

8. The method of claim 1, wherein the single-cell atlas comprises a developmental atlas.

9. The method of claim 8, wherein the single-cell atlas comprises a single-cell organogenesis atlas.

10. The method of claim 8, wherein the single-cell atlas comprises data representing normal development for each of a plurality of mammalian developmental trajectories.

11. The method of claim 10, wherein the single-cell atlas comprises a version of the Mouse Organogenesis Cell Atlas (MOCA).

12. The method of claim 1, wherein the plurality of known biological programs comprises a plurality of known developmental programs.

13. The method of claim 1, further comprising:
(C) applying the machine learning perceptron classifier to the gene expression data for the input tumor to generate the tumor type of the input tumor.

14. The method of claim 13, wherein the input tumor comprises a sample of a cancer of unknown primary.

15. The method of claim 1, wherein using the map to extract, from the plurality of training tumors, the plurality of biological components comprises using the map to deconvolute the plurality of training tumors into the plurality of biological components.

16. The method of claim 1, wherein the developmental stages in the single-cell atlas comprise prenatal developmental stages in the single-cell atlas.

17. A system comprising at least one non-transitory computer-readable medium having computer program instructions stored thereon, the computer program instructions being executable by at least one computer processor to perform a method for using a map which maps from gene expression data for a plurality of training tumors in a tumor atlas to gene expression data representing single cells derived from mammal samples in developmental stages in a single-cell atlas, the method comprising:
(A) using the map to extract, from the plurality of training tumors, a plurality of biological components, thereby generating, for each of the plurality of training tumors and each of the plurality of biological components, a corresponding biological component score representing the training tumor as a compilation of non-gene terms describing a plurality of biological programs; and
(B) constructing, based on the map, a machine learning perceptron classifier that outputs a tumor type of an input tumor based on gene expression data for the input tumor, wherein the input tumor is not among the plurality of training tumors.

18. The system of claim 17, wherein the method further comprises:
(C) before A, generating the map.

19. The system of claim 18, wherein generating the map comprises mapping each of the plurality of training tumors to at least one of the plurality of mammalian developmental trajectories in the single-cell atlas.

20. The system of claim 18, wherein (C) comprises:
(C) (1) receiving the single-cell atlas; and
(C) (2) receiving the tumor atlas;
(C) (3) generating the map based on the single-cell atlas and the tumor atlas.

21. The system of claim 20, wherein the gene expression data for the plurality of training tumors comprises, for each of the plurality of training tumors: (1) gene sequencing data for the training tumor; and (2) a label indicating a type of cancer of the training tumor.

22. The system of claim 20, wherein the gene expression data representing single cells derived from mammal samples in developmental stages in the single-cell atlas comprises gene expression data representing organogenesis of a plurality of mammalian developmental trajectories.

23. The system of claim 17, wherein the tumor atlas comprises a version of The Cancer Genome Atlas (TOGA).

24. The system of claim 17, wherein the single-cell atlas comprises a developmental atlas.

25. The system of claim 24, wherein the single-cell atlas comprises a single-cell organogenesis atlas.

26. The system of claim 24, wherein the single-cell atlas comprises data representing normal development for each of a plurality of mammalian developmental trajectories.

27. The system of claim 26, wherein the single-cell atlas comprises a version of the Mouse Organogenesis Cell Atlas (MOCA).

28. The system of claim 17, wherein the plurality of known biological programs comprises a plurality of known developmental programs.

29. The system of claim 17, further comprising:
(C) applying the machine learning perceptron classifier to the gene expression data for the input tumor to generate the tumor type of the input tumor.

30. The system of claim 29, wherein the input tumor comprises a sample of a cancer of unknown primary.

31. The system of claim 17, wherein using the map to extract, from the plurality of training tumors, the plurality of biological components comprises using the map to deconvolute the plurality of training tumors into the plurality of biological components.

32. The system of claim 17, wherein the developmental stages in the single-cell atlas comprise prenatal developmental stages in the single-cell atlas.

33-56. (canceled)
